(19) United States
(12) Patent Application Publication
(10) Pub. No.: US 2016/0208226 A1
(43) Pub. Date: Jul. 21, 2016

(54) Transaminase Biocatalysts

(71) Applicant: COMMONWEALTH SCIENTIFIC AND INDUSTRIAL RESEARCH ORGANISATION, Campell (AU)

(72) Inventors: Colin Scott, Melba (AU); Matthew Wilding, Canberra (AU); Lars Jermiin, Canberra (AU); Thomas Peat, Carlton North (AU); Janet Newman, Carlton North (AU)

(21) Appl. No.: 14/896,252

(22) PCT Filed: Jun. 12, 2014
(86) PCT No.: PCT/AU2014/000617

§ 371 Date: Dec. 4, 2015

(30) Foreign Application Priority Data
Jun. 12, 2013 (AU) 2013902128

(51) Int. Cl. C12N 9/10

(52) U.S. Cl. CPC C12N 9/1096; C12Y 206/00

(57) ABSTRACT

The present invention relates to pyruvate: ω-amino acid transaminases isolated from a Pseudomonas species. The transaminases act on long chain amino acids and are capable of accepting substrates comprising 8 to 12 carbon atoms. The enzymes are suitable as biocatalysts for the manufacture of nylon.

### FIELD OF THE INVENTION

This application relates to transaminases and methods of using the transaminases as biocatalysts.

### BACKGROUND OF THE INVENTION

Transaminases are increasingly being used for commercial scale manufacture of chemicals, pharmaceuticals and polymers because of their ability to catalyse their reactions with superior regio-, stereo- and enantioselectivity. As proteins, they typically work in aqueous systems, removing the need for volatile, deleterious solvents, and they avoid the production of harmful by-products often associated with competing heavy metal catalysis. Furthermore, biocatalysts can be easily separated from the reaction mixture, and regularly operate at environmentally friendly temperatures and pressures. In essence, they are viewed as being “green” alternatives to conventional catalytic chemistry.

However, biocatalytic process development is often challenged by availability of an enzyme that fulfils the substrate specificity requirement for a given target compound. For example, the long chain amino acids (e.g., C9-12) that are needed for nylon manufacture are not abundant in nature, with no clear physiological role for these molecules.

Because transaminases typically have a limited substrate range, additional transaminases are needed.

### SUMMARY OF THE INVENTION

The present inventors have isolated novel transaminases (such as p6 and p7 from a newly isolated Pseudomonas sp.) which can be used as biocatalysts in the production of industrially relevant including, but not limited to, amines, diamines, and amino acids, all of which have significant applications in, for example, polyamide or polypeptide production.

Accordingly, the present invention provides a substantially purified and/or recombinant polypeptide comprising:

i) an amino acid sequence as provided in any one of SEQ ID NOS:1, 2 or 6 to 12,
ii) an amino acid sequence which is at least 40% identical to any one or more of SEQ ID NOS:1, 2 or 6 to 12, or

iii) a biologically active fragment of i) or ii).

In an embodiment, the present invention provides a substantially purified and/or recombinant polypeptide comprising:

i) an amino acid sequence as provided in SEQ ID NO:1 or SEQ ID NO:2,

ii) an amino acid sequence which is at least 40% identical to i), or

iii) a biologically active fragment of i) or ii).

The present invention also provides a substantially purified and/or recombinant polypeptide comprising:

i) an amino acid sequence as provided in any one of SEQ ID NOS:1, 2 or 6 to 12,

ii) an amino acid sequence which is at least 40% identical to any one or more of SEQ ID NOS: 1, 2 or 6 to 12, or

iii) a biologically active fragment of i) or ii),
wherein the polypeptide has amino transferase activity.

In an embodiment, the present invention also provides a substantially purified and/or recombinant polypeptide comprising:

i) an amino acid sequence as provided in SEQ ID NO: 1 or SEQ ID NO:2,

ii) an amino acid sequence which is at least 40% identical to i), or

iii) a biologically active fragment of i) or ii),
wherein the polypeptide has amino transferase activity.

In an embodiment of the invention, the polypeptide comprises an amino acid sequence which is at least 75%, at least 80%, at least 85%, at least 90%, or at least 95% identical to any one or more of SEQ ID7 NOS:1, 2 or 6 to 12.

In an embodiment of the invention, the polypeptide catalyses the transfer of an amino group from an amino donor to an amino acceptor. In other words, the polypeptide of the invention catalyses removal of an amino group from the amino donor and addition of an amino group to the amino acceptor. As such, both the amino donor and amino acceptor are considered to be “substrates” of the polypeptide.

Transamination reactions are generally reversible. Accordingly, in an embodiment of the invention, the polypeptide catalyses the reversible transfer of an amino group from an amino donor to an amino acceptor.

The inventors have surprising found that the polypeptides of the invention are able to catalyse the deamination/amination of significantly longer substrates, for example, having up to 18 carbons.

Thus, in a further embodiment of the invention, the amino donor or amino acceptor comprises at least 3 carbons. In another embodiment of the invention, the amino donor or amino acceptor comprises at least 4 carbons. In another embodiment of the invention, the amino donor or amino acceptor comprises at least 9 carbons. In a further embodiment of the invention, the amino donor or amino acceptor comprises up to 12, 13, 14, 15, 16, 17, or 18 carbons. In another embodiment of the invention, the amino donor or amino acceptor comprises 9 to 12 carbons. In another embodiment of the invention, the amino donor or amino acceptor comprises 9 to 13 carbons. In another embodiment of the invention, the amino donor or amino acceptor comprises 9 to 14 carbons. In another embodiment of the invention, the amino donor or amino acceptor comprises 9 to 15 carbons. In another embodiment of the invention, the amino donor or amino acceptor comprises 9 to 16 carbons. In another embodiment of the invention, the amino donor or amino acceptor comprises 9 to 17 carbons. In another embodiment of the invention, the amino donor or amino acceptor comprises 9 to 18 carbons.

In an embodiment, the polypeptide comprises:

i) an amino acid sequence as provided in SEQ ID NO:1,

ii) an amino acid sequence which is at least 40% identical to i), or

iii) a biologically active fragment of i) or ii), and
catalyses the transfer of an amino group from an amino donor comprising 3 to 12 carbons to an amino acceptor.

In an embodiment, the polypeptide comprises:

i) an amino acid sequence as provided in SEQ ID NO:1,

ii) an amino acid sequence which is at least 40% identical to i), or

iii) a biologically active fragment of i) or ii), and
catalyses the transfer of an amino group from an amino donor to an amino acceptor comprising 3 to 12 carbons.

In an embodiment, the polypeptide comprises:

i) an amino acid sequence as provided in any one of SEQ ID NOS: 2 or 6 to 12,

ii) an amino acid sequence which is at least 40% identical to any one or more of SEQ ID NOS:2 or 6 to 12, or

iii) a biologically active fragment of i) or ii), and
catalyses the transfer of an amino group from an amino donor comprising 4 to 12 carbons to an amino acceptor.

In an embodiment, the polypeptide comprises:

i) an amino acid sequence as provided in any one of SEQ ID NOS: 2 or 6 to 12.

ii) an amino acid sequence which is at least 40% identical to any one or more of SEQ ID NOS:2 or 6 to 12, or

iii) a biologically active fragment of i) or ii), and
catalyses the transfer of an amino group from an amino donor to an amino acceptor comprising 4 to 12 carbons.

In an embodiment of the invention, the amino donor is an amino acid or an amine compound.

In an embodiment, the amino acid is an ω-amino acid such as, for example, 3-aminopropanoic acid, 4-aminobutyric acid, 5-aminopentanoic acid, 6-aminohexanoic acid, 7-aminoheptanoic acid, 8-aminooctanoic acid, 11-aminoundecanoic acid, 12-aminododecanoic acid, 3-aminoheptanoic acid, 3-aminoisobutyric acid, or a derivative thereof. 3-aminoisobutyric acid is an example of a derivative of 3-aminopropanoic acid or 4-aminobutyric acid.

In an embodiment, the amino acid is a β-amino acid such as, for example, 3-aminoheptanoic acid or a derivative thereof.

In an embodiment, the amine compound is a primary amine such as, for example, 1,4-diaminobutane, 1,5-diaminopentane, 1,6-diaminohexane, 6-aminohexan-1-ol, Taurine, Tyramine, Cyclohexylamine, Isopropylamine, 2-aminoindan, or a derivative thereof.

The primary amine may be a diamine such as, for example, 1,4-diaminobutane, 1,5-diaminopentane, 1,6-diaminohexane, or a derivative thereof.

In an embodiment of the invention, the amine compound is not Diethylaminomalonate, Aminomethylphosphonic acid, 3-aminocyclohexanoic acid, 1-aminocyclohexanoic acid, 5-aminolevulinic acid, 2,6-diaminopimelate, 2,4-diaminobutyrate, Creatine, Citrulline, 2-hydroxy-4-aminobutyrate, Ethylamine, or 1,3-diaminopropane, tert-butylamine.

In an embodiment, the amino acceptor is a carbonyl containing compound such as, for example, a keto acid, a ketone or an aldehyde. The aldehyde may be, for example, glyceraldehyde or gluteraldehyde.

In a further embodiment, the amino acceptor also includes substances which are converted to an amino acceptor by other enzymes or whole cell processes, such as, for example, adipic acid. Adipic acid can be converted by an adipate semialdehyde dehydrogenase to 1,6-hexanedial (adipaldehyde) and 6-oxohexanoic acid (adipate semialdehyde) both of which can serve as amino acceptors.

In an embodiment, the polypeptide of the invention has a pH optimum of about pH 10.

In an embodiment of the invention, the polypeptide is fused to at least one other polypeptide. The at least one other polypeptide may be, for example, a polypeptide that enhances the stability of a polypeptide of the present invention, or a polypeptide that assists in the purification of the fusion protein.

The present invention also provides an isolated and/or exogenous polynucleotide comprising one or more of:

i) a sequence of nucleotides as provided in any one of SEQ ID NOs:3 to 5, or 14 to 20.

ii) a sequence of nucleotides encoding a polypeptide of the invention,

iii) a sequence of nucleotides which is at least 45% identical to any one or more of SEQ ID NOs:3 to 5, or 14 to 20,

iv) a sequence of nucleotides which hybridizes to i) under stringent conditions, or

v) a sequence of nucleotides complementary to any one of i) to iv).

In an embodiment, the polynucleotide comprises one or more of:

i) a sequence of nucleotides as provided in SEQ ID NO:3, SEQ ID NO:4. or SEQ ID NO:5,

ii) a sequence of nucleotides encoding a polypeptide of any one of claims 1 to 13,

iii) a sequence of nucleotides which is at least 45% identical to i),

iv) a sequence of nucleotides which hybridizes to i) under stringent conditions, or

v) a sequence of nucleotides complementary to any one of i) to iv).

Preferably, the polynucleotide encodes a polypeptide which encodes a polypeptide that has amino transferase activity.

Preferably, the polynucleotide is operably linked to a promoter.

In an embodiment of the invention, the polypeptide comprises an amino acid sequence which is at least 65%, at least 70%, at least 75%, at least 80%. at least 85%, at least 90%, or at least 95% identical to any one or more of SEQ ID NOs:3 to 5, or 14 to 20.

The present invention provides a vector comprising a polynucleotide of the invention. Preferably, the polynucleotide is operably linked to a promoter.

The present invention also provides a host cell comprising a polynucleotide of the invention or a vector of the invention.

The host cell can be any type of cell. In an embodiment of the invention, the host cell is a bacterial or fungal cell.

The present invention also provides a method for producing a polypeptide of the invention, the method comprising cultivating a host cell of the invention encoding said polypeptide, or a vector of the invention encoding said polypeptide in a cell free expression system, under conditions which allow expression of the polynucleotide encoding the polypeptide, and recovering the expressed polypeptide.

Also provided is a polypeptide produced using a method of the invention.

The present invention also provides an isolated or purified antibody which specifically binds to a polypeptide of the invention.

The present invention also provides a transgenic non-human organism, for example, a transgenic non-human animal or plant, comprising an exogenous polynucleotide encoding at least one polypeptide of the invention.

Preferably, the polynucleotide is stably incorporated into the genome of the organism.

The present invention also provides an extract of a host cell of the invention, or a transgenic non-human organism of the invention, wherein the extract comprises a polypeptide of the invention.

The present invention also provides a composition comprising one or more of all of: a polypeptide of the invention, a polynucleotide of the invention, a vector of the invention, a host cell of the invention, an antibody of the invention, a transgenic non-human organism of the invention, or an extract of the invention.

The present invention also provides a method for catalysing transfer of an amino group from an amino donor to an amino acceptor, the method comprising contacting the amino donor and amino acceptor with a polypeptide of the invention or a composition of the invention. The polypeptide may be produced by a host cell of the invention.

In a further embodiment of the invention, the amino donor or amino acceptor comprises at least 3 carbons. In another embodiment of the invention, the amino donor or amino acceptor comprises at least 4 carbons. In another embodiment of the invention, the amino donor or amino acceptor comprises at least 9 carbons. In a further embodiment of the invention, the amino donor or amino acceptor comprises up to 12, 13, 14, 15, 16, 17, or 18 carbons. In another embodiment of the invention, the amino donor or amino acceptor comprises 9 to 12 carbons. In another embodiment of the invention, the amino donor or amino acceptor comprises 9 to 13 carbons. In another embodiment of the invention, the amino donor or amino acceptor comprises 9 to 14 carbons. In another embodiment of the invention, the amino donor or amino acceptor comprises 9 to 15 carbons. In another embodiment of the invention, the amino donor or amino acceptor comprises 9 to 16 carbons. In another embodiment of the invention, the amino donor or amino acceptor comprises 9 to 17 carbons. In another embodiment of the invention, the amino donor or amino acceptor comprises 9 to 12, 9 to 13, 9 to 14 9 to 15, 9 to 16, 9 to 17, or 9 to 18 carbons.

The present invention also provides a method for catalysing transfer of an amino group from an amino donor to an amino acceptor with a substantially purified and/or recombinant polypeptide which has amino transferase activity, wherein the amino donor or amino acceptor comprises at least 9 carbons. In one embodiment, both the amino donor and amino acceptor have at least 9 carbons. In an embodiment, the amino donor or amino acceptor comprises 9 to 12, 9 to 13, 9 to 14 9 to 15, 9 to 16, 9 to 17, or 9 to 18 carbons. The polypeptide may be a polypeptide of the invention. The polypeptide may be produced by a host cell of the invention.

The above methods may further comprise adding a cofactor, for example, pyridoxal 5′ phosphate (PLP), PLP-pyridoxal phosphate, or pyridoxamine phosphate. In a preferred embodiment, the cofactor is PLP-pyridoxal phosphate.

In an embodiment, the methods produce an industrial product.

In a further embodiment, the methods further comprise one or more reactions to produce an industrial product. For example, the deaminated amino donor or the aminated amino acceptor can be further reacted, for example, with one or more enzymes of compounds to produce an industrial product.

The industrial product may be one or more or all of: an amino acid, diacid, amine, diamine, keto acid, diketo acid, ketone, diketone, aldehyde, dialdehyde, semialdehyde, amino aldehyde, polypeptide, polyamine, polyamide, polyketone, polyaldehyde, lactam, lactone, or fatty acid.

In an embodiment, the industrial product is an amino acid, for example, an ω-amino acid such as, for example, 3-aminopropanoic acid, 4-aminobutyric acid, 5-aminopentanoic acid, 6-aminohexanoic acid, 7-aminoheptanoic acid, 3-aminoheptanoic acid, 8-aminooctanoic acid, 11-aminoundecanoic acid, 12-aminododecanoic acid, 3-aminoheptanoic acid, 3-aminoisobutyric acid, or a derivative thereof. In another embodiment, the industrial product is a β-amino acid such as, for example, 3-aminoheptanoic acid or a derivative thereof

In another embodiment, the industrial product is an amine such as, for example, 1,4-diaminobutane, ,5 -diaminopentane, 1,6-diaminohexane, 6-aminohexan-1 -ol, Taurine, Tyramine, Cyclohexylamine, Isopropylamine, 2-aminoindan, or a derivative thereof.

In another embodiment, the industrial product is a compound comprising a carbonyl group such as, for example, a keto acid, a ketone or an aldehyde. The aldehyde may be, for example, glyceraldehyde or gluteraldehyde.

In another embodiment, the industrial product is a polyamide such as for example, nylon.

In another embodiment, the industrial product is a lactam.

In another embodiment, the industrial product is a polypeptide.

In another embodiment, the industrial product is a functionalised fatty acid.

The methods of the invention can be used to produce pharmaceutically relevant industrial products, for example, pharmaceutically relevant amines, polyamines, amino acids including ω-amino acids, polypeptides and lactams.

The methods of the invention can also be used in the production of polyamides such as nylon. For example, the polypeptides of the invention when used in conjunction with an adipate semialdehyde dehydrogenase, can be used to produce the diacid and diamine components of nylon 6,6 (shown below left), or alternatively the co-amino acid that makes nylon 6 (below right), from adipic acid.

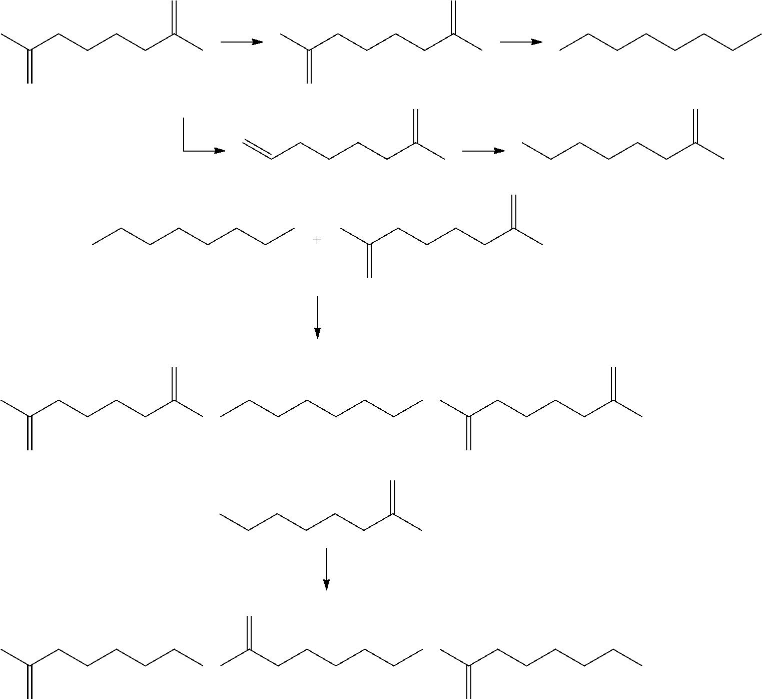

In this example, the diamine (hexamethylenediamine) and the ω-amino acid (aminocaproic acid) can be considered industrial products as can nylon 6,6 and nylon 6.

The methods of the invention can also be used to produce industrial products from amino acceptors. For example, glycerol (bulk diesel waste product) derivatives such as glyceraldehydes, dihydroxyacetone or ketomalonates can be used as “substrates” for chemical production.

The methods of the invention can also be used to functionalise fatty acids. Aliphatic fatty acids can function as amino acceptors in the methods of the invention

It will also be recognised that the amino donors and amino acceptors may possess asymmetric centres and are therefore capable of existing in more than one stereoisomeric form. The invention thus also relates to the use and production of the amino donors and amino acceptors in substantially pure isomeric form at one or more asymmetric centres, for example, greater than about 90% ee, such as about 95% or 97% ee or greater than 99% ee, as well as mixtures, including racemic mixtures, thereof. Such isomers may be prepared by asymmetric synthesis, for example using chiral intermediates, or by chiral resolution.

In an embodiment, the methods of the invention can also be used to make enatiopure amines. Chiral amines in enatiopure forms are important chemical building blocks, which may be used in for example, the production of pharmaceuticals.

The polypeptides of the present invention can be mutated, and the resulting mutants screened for altered activity, such as enhanced enzymatic activity or altered substrate specificity. Such mutations can be performed using any technique known in the art including, but not limited to, site-saturated mutagenesis.

Thus, the present invention also provides a method of producing a polypeptide with enhanced ability to catalyse the transfer of an amino group from an amino donor to an amino acceptor, or having altered substrate specificity, the method comprising:

i) altering one or more amino acids of a polypeptide of the invention,

ii) determining the ability of the altered polypeptide obtained from step i) to catalyse the transfer of an amino group from the amino donor to the amino acceptor, and

iii) selecting an altered polypeptide with enhanced ability to catalyse the transfer of an amino group from the amino donor, or having altered substrate specificity, when compared to the polypeptide used in step i).

Step i) can be performed using any suitable technique known in the art such as, but not limited to, site-directed mutagenesis, chemical mutagenesis and DNA shuffling of the encoding nucleic acid.

Also provided is a polypeptide produced by a method of the invention.

The present invention also provides a method for screening for a microorganism capable of catalysing the transfer of an amino group from an amino donor which comprises at least 9 carbons, the method comprising:

i) culturing a candidate microorganism in the presence of an amino donor which comprises at least 9 carbons, as a sole nitrogen source, and

ii) determining whether the microorganism is capable of growth and/or division.

In an embodiment, the amino donor comprises 9 to 12, 9 to 13, 9 to 14 9 to 15, 9 to 16, 9 to 17, or 9 to 18 carbons.

Also provided is a microorganism identified using a method of the invention.

The present invention also provides a kit comprising one or more or all of: a polypeptide of the invention, a polynucleotide of the invention, a vector of the invention, a host cell of the invention, an antibody of the invention, a transgenic non-human organism of the invention or an extract of the invention.

The present invention also provides a crystal structure of an amino transferase of the invention. In an embodiment, the amino transferase has an amino acid sequence as provided in SEQ ID NO:1 or SEQ ID NO:2.

The present invention also provides a set of atomic coordinates, or subset thereof, of a crystal structure of the invention.

The present invention also provides a set of atomic coordinates, or subset thereof, provided in Appendix I.

The present invention also provides a computer-readable medium having recorded thereon data representing the atomic coordinates, or subset thereof, of a crystal structure of the invention; or the atomic coordinates, or subset thereof, provided in Appendix I; and/or a model produced using the atomic coordinates.

The present invention also provides a computer-assisted method of identifying a compound that binds to the amino transferase of the invention, the method comprising the steps of:

i) docking the structure of a candidate compound to a structure defined by the atomic coordinates, or subset thereof, of a crystal structure of the invention; or the atomic coordinates, or subset thereof, provided in Appendix I, and

ii) identifying a candidate compound which may bind to the amino transferase.

In an embodiment, the amino transferase has an amino acid sequence as provided in SEQ ID NO:1 or SEQ ID NO:2.

In an embodiment, the method further comprises synthesising or obtaining an identified candidate compound and determining if the compound binds to the amino transferase.

The present invention also provides a computer-assisted method for identifying a polypeptide which has amino transferase activity, the method comprising the steps of:

i) comparing a structure defined by the atomic coordinates, or subset thereof, of a crystal structure of the invention; or the atomic coordinates, or subset thereof, provided in Appendix I, to a model of the tertiary structure of a candidate polypeptide, and

ii) identifying a candidate compound which may have amino transferase activity.

In an embodiment, the method further comprises synthesising or obtaining an identified polypeptide and determining if the polypeptide catalyses the transfer of an amino group from an amino donor to an amino acceptor.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein

The invention is hereinafter described by way of the following non-limiting Examples and with reference to the accompanying Figures.

### BRIEF DESCRIPTION OF THE ACCOMPANYING DRAWINGS

FIG. 1. Alignment of polypeptides produced using ancestral reconstruction. KEY TO SEQUENCE LISTING

SEQ ID NO:1: Amino acid sequence of p6.

SEQ ID NO:2: Amino acid sequence of p7.

SEQ ID NO:3: Nucleotide sequence of p6.

SEQ ID NO:4: Nucleotide sequence of p7.

SEQ ID NO:5: Nucleotide sequence of p7 having a single point mutation.

SEQ ID NO:6: Amino acid sequence of p4.

SEQ ID NO:7: Amino acid sequence of p7N6.

SEQ ID NO:8: Amino acid sequence of p7N15.

SEQ ID NO:9: Amino acid sequence of p7N16.

SEQ ID NO:10: Amino acid sequence of p7N17.

SEQ ID NO:11: Amino acid sequence of p7N43.

SEQ ID NO:12: Amino acid sequence of p7N48.

SEQ ID NO:13: Amino acid sequence of GabT (Gamma-aminobutyrate Transaminase; E.C. 2.6.1.19).

SEQ ID NO:14: Nucleotide sequence of p4.

SEQ ID NO:15: Nucleotide sequence of p7N6.

SEQ ID NO:16: Nucleotide sequence of p7N15.

SEQ ID NO:17: Nucleotide sequence of p7N16.

SEQ ID NO:18: Nucleotide sequence of p7N17.

SEQ ID NO:19: Nucleotide sequence of p7N43.

SEQ ID NO:20: Nucleotide sequence of p7N48.

### DETAILED DESCRIPTION OF THE PREFERRED EMBODIMENTS

### General Techniques and Definitions

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, polypeptide and polyamide production, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), Maniatis et al., Molecular Cloning: A Laboratory Manual, Cold Spring Harbour Laboratory Press (1982), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), F. M. Ausubel et al., (editors), Current Protocols in Molecular Biology, Greene Pub. Associates and Wiley-Interscience (1988, including all updates until present), E. Harlow and D. Lane (editors), Antibodies: A Laboratory Manual. Cold Spring Harbour Laboratory (1988), and J. E. Coligan et al. (editors), Current Protocols in Immunology, John Wiley & Sons (1991, including all updates until present).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, “about” shall generally mean within 20%, more preferably within 10%, and even more preferably within 5%, of a given value or range.

The term “and/or”, e.g., “X and/or Y” shall be understood to mean either “X and Y” or “X or Y” and shall be taken to provide explicit support for both meanings or for either meaning.

### Polypeptides

The present invention relates to polypeptides which catalyse transfer of an amino group from an amino donor to an amino acceptor. Examples of such polypeptides include, but are not limited to, those comprising an amino acid sequence as provided in any one of SEQ ID NOs:1, 2 or 6 to 12.

The terms “polypeptide” and “protein” are generally used interchangeably.

A polypeptide or class of polypeptides may be defined by the extent of identity (% identity) of its amino acid sequence to a reference amino acid sequence, or by having a greater % identity to one reference amino acid sequence than to another. The % identity of a polypeptide to a reference amino acid sequence is typically determined by GAP analysis (Needleman and Wunsch, 1970; GCG program) with parameters of a gap creation penalty=5, and a gap extension penalty=0.3. The query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the query sequence is at least 450 amino acids in length and the GAP analysis aligns the two sequences over a region of at least 450 amino acids. Even more preferably, the GAP analysis aligns two sequences over their entire length. The polypeptide or class of polypeptides may have the same enzymatic activity as, or a different activity than, or lack the activity of, the reference polypeptide. Preferably, the polypeptide has an enzymatic activity of at least 10%, at least 50%, at least 75%, or at least 90%, of the activity of the reference polypeptide.

As used herein a “biologically active fragment” is a portion of a polypeptide of the invention which maintains a defined activity of a full-length reference polypeptide namely, amino transferase activity. Biologically active fragments as used herein exclude the full-length polypeptide. Biologically active fragments can be any size portion as long as they maintain the defined activity. Preferably, the biologically active fragment maintains at least 10%, at least 50%, at least 75%, or at least 90%, of the activity of the full length protein.

With regard to a defined polypeptide or enzyme, it will be appreciated that % identity figures higher than those provided herein will encompass preferred embodiments. Thus, where applicable, in light of the minimum % identity figures, it is preferred that the polypeptide/enzyme comprises an amino acid sequence which is at least 40%, more preferably at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Amino acid sequence mutants of the polypeptides defined herein can be prepared by introducing appropriate nucleotide changes into a nucleic acid defined herein, or by in vitro synthesis of the desired polypeptide. Such mutants include for example, deletions, insertions, or substitutions of residues within the amino acid sequence. A combination of deletions, insertions and substitutions can be made to arrive at the final construct, provided that the final polypeptide product possesses the defined activity. Preferred amino acid sequence mutants have only one, two, three, four or less than 10 amino acid changes relative to the reference polypeptide.

Mutant (altered) polypeptides can be prepared using any technique known in the art, for example, using directed evolution or rational design strategies (see below). Products derived from mutated/altered DNA can readily be screened using techniques described herein to determine if they possess amino transferase activity.

In designing amino acid sequence mutants, the location of the mutation site and the nature of the mutation will depend on characteristic(s) to be modified. The sites for mutation can be modified individually or in series for example, by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide removed and a different residue inserted in its place. The sites of greatest interest for substitutional mutagenesis include sites identified as the active site(s) for example substrate or co-factor binding sites. Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1 under the heading of “exemplary substitutions”.

In a preferred embodiment a mutant/variant polypeptide has only, or not more than, one or two or three or four conservative amino acid changes when compared to a reference polypeptide. Details of conservative amino acid changes are provided in Table 1. As the skilled person would be aware, such minor changes can reasonably be predicted not to alter the activity of the polypeptide when expressed in a cell.

TABLE 1
Exemplary substitutions

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

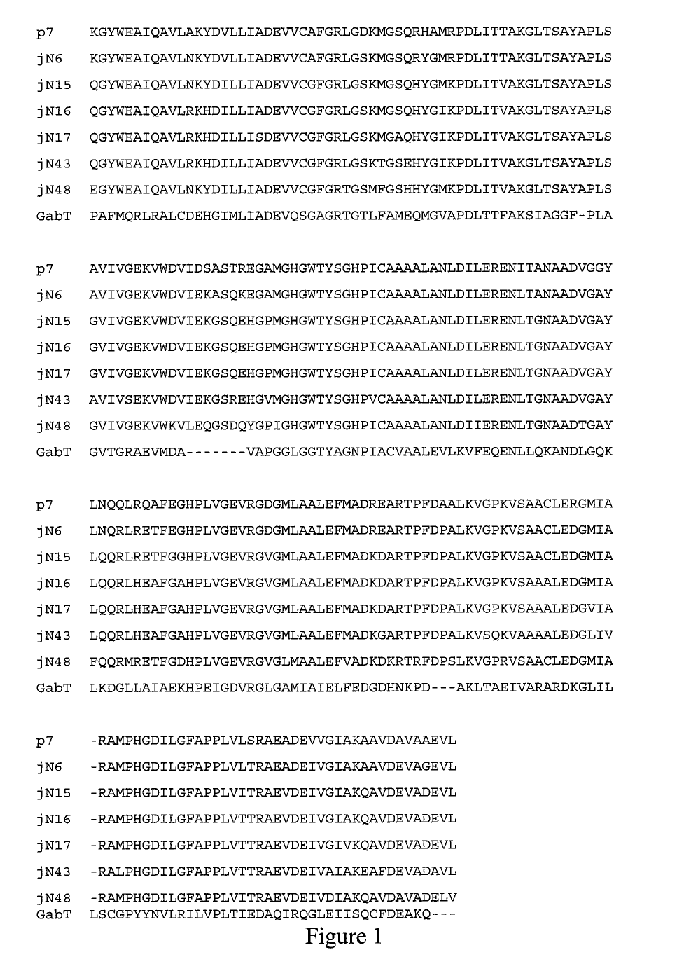

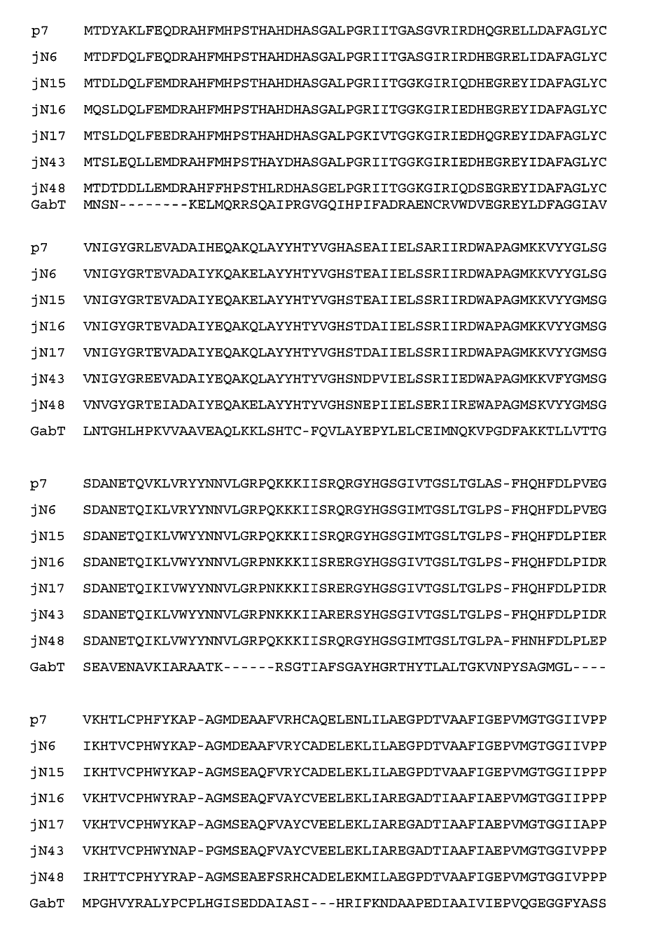

Considerable guidance regarding amino acid subsitutions which can be made can be obtained by aligning different amino acid transferases described herein (see, for example, FIG. 1). Such alignments are highly informative regarding which amino acids can be altered, and if so what amino acids can be used at a particular site and function maintained.

In an embodiment, amino acids in the cofactor/substrate binding site are not altered, or if they are it is with a conservative amino acid substitution. Examples of amino acids in the cofactor/substrate binding site are outlined below;

p6: F89, V320, G325, T327, F24, L57, L60, W61, Y153, I166, G168, K171, S231, K288, I396, R414, F415, G416, G417, Q421, V156,

p7: S19, L58, Y59, H85, Y87, V88, L118, S119, G120, S121, Y153, G155, F169, E226, T231, D259, V261, V262, A287, K288, L297, A319, H322. G323, W324, T325, Y326, R419,

p7N6: S19, L58, Y59, H85, Y87, V88, L118, S119, G120, S121, Y153, G155, F169, E226, T231, D259, V261, V262, A287, K288, L297, A319, H322, G323, W324, T325, Y326, R419,

p7N15: S19, L58, Y59, H85, Y87, V88, L118, S119, G120, S121, Y153, G155, F169, E226, T231, D259, V261, V262, A287, K288, L297, P319, H322, G323, W324, T325, Y326, R419,

p7N16: S19, L58, Y59, H85, Y87, V88, M118, S119, G120, S121, Y153, G155,

F169, E226, T231, D259, V261, V262, A287, K288, L297, P319, H322, G323, W324, T325. Y326, R419,

p7N17: S19, L58, Y59, H85, Y87, V88, M118, S119, G120, S121, Y153, G155, F169, E226, T231, D259, V261, V262, A287, K288, L297, P319, H322, G323, W324, T325, Y326, R419

p7N43: S19, L58, Y59, H85, Y87, V88, M118, S119, G120, S121, Y153, G155, F169, E226, T231, D259, V261, V262, A287, K288, L297, V319, H322, G323, W324, T325, Y326, R419, and

p7N48: S19, L58, Y59, H85, Y87, V88, M118, S119, G120, S121, Y153, G155, F169, E226, T231, D259, V261, V262, A287, K288, L297, P319, H322, G323, W324, T325, Y326, R419.

Furthermore, if desired, unnatural amino acids or synthetic amino acid analogues can be introduced as a substitution or addition into the polypeptides of the invention. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general.

Also included within the scope of the invention are polypeptides which are differentially modified during or after synthesis, for example, by biotinylation, benzylation, glycosylation, acetylation, phosphorylation, amidation, derivatization by known protecting/blocking groups, proteolytic cleavage, linkage to an antibody molecule or other cellular ligand. etc. These modifications may serve to increase the stability and/or bioactivity of the polypeptides of the invention.

Polypeptides of the present invention can be produced in a variety of ways, including production and recovery of natural polypeptides, production and recovery of recombinant polypeptides, and synthetic synthesis of the polypeptides. In an embodiment, an isolated polypeptide of the present invention is produced by culturing a cell capable of expressing the polypeptide under conditions effective to produce the polypeptide, and recovering the polypeptide. A preferred cell to culture is a host cell of the present invention. Effective culture conditions include, but are not limited to, effective media, bioreactor, temperature, pH and oxygen conditions that permit polypeptide production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins.

Cells of the present invention can be cultured in conventional fermentation bioreactors, shake flasks, test tubes, microtiter dishes, and petri plates. Culturing can be carried out at a temperature, pH and oxygen content appropriate for a host cell. Such culturing conditions are within the expertise of one of ordinary skill in the art.

### Directed Evolution

In directed evolution, random mutagenesis is applied to a protein, and a selection regime is used to pick out variants that have the desired qualities, for example, increased amino transferase activity. Further rounds of mutation and selection are then applied. A typical directed evolution strategy involves three steps:

1) Diversification: The gene encoding the protein of interest is mutated and/or recombined at random to create a large library of gene variants. Variant gene libraries can be constructed through error prone PCR (see, for example, Leung, 1989; Cadwell and Joyce, 1992), from pools of DNaseI digested fragments prepared from parental templates (Stemmer, 1994a; Stemmer, 1994b; Crameri et al., 1998; Coco et al., 2001) from degenerate oligonucleotides (Ness et al., 2002, Coco, 2002) or from mixtures of both, or even from undigested parental templates (Zhao et al., 1998; Eggert et al., 2005; Jezequek et al., 2008) and are usually assembled through PCR. Libraries can also be made from parental sequences recombined in vivo or in vitro by either homologous or non-homologous recombination (Ostermeier et al., 1999; Volkov et al., 1999; Sieber et al., 2001). Variant gene libraries can also be constructed by sub-cloning a gene of interest into a suitable vector, transforming the vector into a “mutator” strain such as the E. coli XL-1 red (Stratagene) and propagating the transformed bacteria for a suitable number of generations. Variant gene libraries can also be constructed by subjecting the gene of interest to DNA shuffling (i.e., in vitro homologous recombination of pools of selected mutant genes by random fragmentation and reassembly) as broadly described by Harayama (1998).

2) Selection: The library is tested for the presence of mutants (variants) possessing the desired property using a screen or selection. Screens enable the identification and isolation of high-performing mutants by hand, while selections automatically eliminate all nonfunctional mutants. A screen may involve screening for the presence of known conserved amino acid motifs. Alternatively, or in addition, a screen may involve expressing the mutated polynucleotide in a cell or transgenic non-human organism or part thereof and assaying the level of, for example, amino transferase activity by, for example, quantifying the level of resultant product in the cell or transgenic non-human organism or part thereof or extracted from the cell or transgenic non-human organism or part thereof, and determining the level of product relative to a corresponding cell or transgenic non-human organism or part thereof lacking the mutated polynucleotide and optionally, expressing the parent (unmutated) polynucleotide. Alternatively, the screen may involve feeding the cell or transgenic non-human organism or part thereof labelled substrate and determining the level of substrate or product in the cell or transgenic non-human organism or part thereof, or extracted from the cell or transgenic non-human organism or part thereof relative to a corresponding cell or transgenic non-human organism or part thereof lacking the mutated polynucleotide and optionally, expressing the parent (unmutated) polynucleotide.

3) Amplification: The variants identified in the selection or screen are replicated many fold, enabling researchers to sequence their DNA in order to understand what mutations have occurred.

Together, these three steps are termed a “round” of directed evolution. Most experiments will entail more than one round. In these experiments. the “winners” of the previous round are diversified in the next round to create a new library. At the end of the experiment, all evolved protein or polynucleotide mutants are characterized using biochemical methods.

Rational Design

A protein can be designed rationally, on the basis of known information about protein structure and folding. This can be accomplished by design from scratch (de novo design) or by redesign based on native scaffolds (see, for example, Hellinga, 1997; and Lu and Berry, Protein Structure Design and Engineering, Handbook of Proteins 2, 1153-1157 (2007)). Protein design typically involves identifying sequences that fold into a given or target structure and can be accomplished using computer models. Computational protein design algorithms search the sequence-conformation space for sequences that are low in energy when folded to the target structure. Computational protein design algorithms use models of protein energetics to evaluate how mutations would affect a protein's structure and function. These energy functions typically include a combination of molecular mechanics, statistical (i.e., knowledge-based), and other empirical terms. Suitable available software includes IPRO (Interative Protein Redesign and Optimization), EGAD (A Genetic Algorithm for Protein Design), Rosetta Design, Sharpen and Abalone.

Amino Transferase Activity

The polypeptides of the invention are transaminases (also referred to herein as amino transferases).

As used herein, the term “transaminase” or “amino transferase” refers to an enzyme which catalyses the transfer of an amino group from an amino donor to an amino acceptor, for example, from an amino acid to an α-keto acid. This enzyme class can be divided into four subgroups based on sequence alignment (Mehta et al., 1993). Enzymes in subgroups I, III and IV transfer the amino group bonded to an α-carbon of amino acids. Transaminases in subgroup II can transfer the amino group from a carbon atom that does not bear a carboxyl group. Transaminases in subgroup II are often referred to as ω-amino acid transaminases. In a preferred embodiment, the polypeptides of the invention are ω-amino acid transaminases For a review of ω-amino acid transaminases see Malik et al. (2012).

As used herein “ω-amino acid transaminases” refers to transaminases that catalyse transfer of a non a-amino group from an amino donor to an amino acceptor. Such transferases typically have greater substrate specificity and may also be capable of transferring an amino group from an amine compound to a compound comprising a carbonyl group, for example, to a keto acid, a ketone, or an aldehyde.

As used herein, the term “ω-amino acid” refers to a compound having an amino or amine group (NH₂) and a carboxyl group (COOH) in which the amino group and the carboxyl group are separated by a single carbon atom, the ω-carbon atom. An ω-amino acid includes naturally occurring and non-naturally occurring L-amino acids and their D-isomers.

As used herein, the term “ω-amino acid” refers to amino acids which have the amino group attached to a non α-carbon. The term is a general term that does not specify the actual position of the amino group but represents all non-α positions and therefore covers, for example, β-, γ- and δ-amino acids.

As used herein, the term “β-amino acid” refers to an amino acid that differs from an α-amino acid in that there are two (2) carbon atoms separating the carboxyl terminus and the amino terminus. The β-amino acid may be, for example, β-aminoheptanoic acid or a derivative thereof. β-amino acids with a specific side chain can exist as the R or S enantiomers at either of the α (C2) carbon or the β (C3) carbon, resulting in a total of 4 possible isomers for any given side chain (as shown below). The side chains may be the same as those of naturally occurring α-amino acids or may be the side chains of non-naturally occurring amino acids.

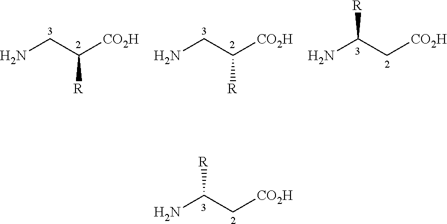

Similarly, the chiral carbons of other ω-amino acids can exist as the R or S enantiomers. As would be understood in the art, as the number of carbon atoms separating the carboxyl terminus and the amino terminus increases the number of possible isomeric forms also increases. For example, if a molecule contains two asymmetric carbons, there are up to 4 possible configurations. The possibilities continue to multiply as there are more asymmetric centers in a molecule. In general, the number of configurational isomers of a molecule can be determined by calculating 2ⁿ, where n=the number of chiral centers in the molecule. This holds true except in cases where the molecule has meso forms.

The “amino donor” may be any compound capable of donating an amino or amine group (NH₂), for example. an amino acid or an amine compound. Amino donors include the non-chiral amino acid glycine, chiral amino acids having the S-configuration such as L-alanine or L-aspartic acid, ω-amino acids such as 3-aminopropanoic acid (β-alanine), 4-aminobutyric acid, 5-aminopentanoic acid, 6-aminohexanoic acid, 7-aminoheptanoic acid, 8-aminooctanoic acid, 11-aminoundecanoic acid, 12-aminododecanoic acid, 3 -aminoheptanoic acid, 3-aminoisobutyric acid and derivatives thereof. Amino donors, however, need not be amino acids. For example, amines such as 6-aminohexan-1-ol, Taurine, Tyramine, Cyclohexylamine, Isopropylamine, 2-aminoindan, and derivatives thereof, and diamines such as 1,4-diaminobutane, 1,5-diaminopentane, and 1,6-diaminohexane, and derivatives thereof can be used as amino donors. The amino donor may be a compound which is converted to an amino donor by an enzyme or whole cell process(es).

The carbon atoms of the amino donor can be arranged as a straight alkyl chain, a branched alkyl chain, cycloalkyl groups and aryl groups or a combination thereof.

The term “cycloalkyl” as used herein, refers to cyclic hydrocarbon groups. Suitable cycloalkyl groups include, but are not limited to, cyclopropyl, cyclobutyl, cyclopentyl and cyclohexyl.

The term “aryl” as used herein, refers to a C₆-C₁₀ aromatic hydrocarbon group, for example phenyl or naphthyl.

An “amino acid” is a compound that comprises an amino or amine (NH₂) group and a carboxyl group (COOH) Amino acids having two (2) amine groups and at least one carboxyl group can be more specifically referred to as “diamino acids”. However, as used herein, the term “amino acid” will be generally understood to include diamino acids. The stereo configuration of the α-carbon is typically referred to using the D/L notation with reference to the absolute configuration of Glyceraldehyde. Alternatively. (S) and (R) designators may be used to indicate the absolute stereochemistry.

The term “non-naturally occurring amino acid” as used herein, refers to amino acids having a side chain that does not occur in nature. Examples of non-natural amino acids and derivatives include, but are not limited to, 4-aminobutyric acid, 5-aminopentanoic acid, 6-aminohexanoic acid, 7-aminoheptanoic acid, 8-aminooctanoic acid, 11-aminoundecanoic acid, 12-aminododecanoic acid, 3-aminoheptanoic acid, and 3-aminoisobutyric acid.

An “amine compound” is a compound (including straight chain, branched and cyclic compounds) that comprises a nitrogen atom connected by three sigma bonds to alkyl, aryl or alkylaryl groups or hydrogen atoms. Amines are classified as primary, secondary, or tertiary according to the number of carbons bonded directly to the nitrogen atom. Primary amines have one carbon bonded to the nitrogen. Secondary amines have two carbons bonded to the nitrogen, and tertiary amines have three carbons bonded to the nitrogen. The amine compound may have one or more primary amino groups (NH₂). Amines having two primary amino groups can be more specifically referred to as “diamines”. Amines having two or more primary amino groups can be more specifically referred to as polyamines. However, as used herein, the term “amine” will be generally understood to include diamines and polyamines. An amine compound may be a chiral amine compound.

The “amino acceptor” may be any compound capable of accepting an amino or amine group (NH₂), for example, a compound comprising a carbonyl group (C═O), for example, a keto acid, a ketone, or an aldehyde. The amino acceptor may also be a compound which is converted to an amino acceptor by an enzyme or whole cell process(es), such as fumaric acid (which can be converted to oxaloacetic acid), or glucose (which can be converted to pyruvate), or adipic acid (which can be converted by an adipate semialdehyde dehydrogenase to 1,6-hexanedial (adipaldehyde) and 6-oxohexanoic acid (adipate semialdehyde).

A “carbonyl group” is a functional group composed of a carbon atom double bonded to an oxygen atom (C═O).

A “keto acid (or oxacid)” is a compound that comprises a carboxylic acid group (—COOH) and a ketone group (R(CO)R¹). Keto acids include, for example, glyoxalic acid, pyruvic acid, oxaloacetic acid, and the like, as well as salts of these acids. Keto acids having two or more ketone groups and at least one carboxylic acid group can be more specifically referred to as “diketo acids”.

A “ketone (or alkanone)” is a compound that comprises a carbonyl group bonded to two other carbon atoms having the general formula R(CO)R¹, where R may be the same or different to R¹. Ketones having two ketone groups can be more specifically referred to as diketones. Ketones having two or more ketone groups can be more specifically referred to as polyketones. However, as used herein, the term “ketone” will be generally understood to include diketones and polyketones.

An “aldehyde” is a compound comprising a formyl or aldehyde group (CHO) having the general formula R—CHO. The formyl group consists of a carbonyl (C═O) center bonded to hydrogen and an R group, which is H or alkyl or aryl or arylalkyl. Aldehydes differ from ketones in that the carbonyl is placed at the end of a carbon skeleton rather than between two carbon atoms. Aldehydes include, for example, glutaraldehyde (pentanedial) and glyceraldehyde. Aldehydes having two formyl groups can be more specifically referred to as “dialdehydes”. Aldehydes having two or more formyl groups can be more specifically referred to as “polyaldehydes”. Aldehydes having a formyl group and a carboxyl group (COOH) can be more specifically referred to as “semialdehydes”. Aldehydes having a formyl group an amino or amine group (NH₂) can be more specifically referred to as “aminoaldehydes”. However, as used herein, the term “aldehyde” will be generally understood to include dialdehydes, polyaldehydes semialdehydes and aminoaldehydes.

As used herein, “amino transferase activity” refers to the ability of a polypeptide to catalyse transfer of an amino group from an amino donor to an amino acceptor. Typically, the polypeptide can catalyse reversal of the transamination. The transferase reaction is divided into two half reactions: oxidative deamination of an amino donor and reductive amination of an amino acceptor. For activity, a co-factor is required and acts as an intermediate carrier of the amino group during the reaction.

In an embodiment, the polypeptide comprises:

i) an amino acid sequence as provided in SEQ ID NO:1,

ii) an amino acid sequence which is at least 40% identical, or at least 80% identical, or at least 90% identical, or at least 95% identical, to SEQ ID NO:1, or

iii) a biologically active fragment of i) or ii).

wherein the polypeptide has amino transferase activity on one, more or all of the substrates selected from, but not limited to glycine, beta-alanine, 4-aminobutyrate, 5-aminopentanoate, 6-aminohexanoic acid, 7-aminoheptanoic acid, 8-aminooctanoic acid, 11-aminoundecanoic acid. 12-aminododecanoic acid, 3-aminoisobutyrate, putrescine, cadaverine, 3-aminocyclohexanoate, propionaldehyde, butyraldehyde, tyramine, 2-aminoindan, hexamethylenediamine, 1,7-diaminoheptane, 1,8-diaminooctane, 1,9-diaminononane, 1,10-diaminodecane, 6-aminohexanol, 7-aminoheptanol, 8-aminooctanol, 9-aminononanol, 10-aminodecanol, taurine, glyceraldehyde, 3-aminoheptanoic acid, cyclohexylamine, 2-methylbenzylamine, dihydroxyacetone phosphate, hydroxymethylfurfural, ethanolamine, alanine and pyruvate.

In an embodiment, the polypeptide comprises:

i) an amino acid sequence as provided in SEQ ID NO:2,

ii) an amino acid sequence which is at least 40% identical, or at least 80% identical, or at least 90% identical, or at least 95% identical, to SEQ ID NO:2, or

iii) a biologically active fragment of i) or ii).
wherein the polypeptide has amino transferase activity on one, more or all of the substrates selected from, but not limited to, glycine, 4-aminobutyrate, 5-aminopentanoate, 6-aminohexanoic acid, 7-aminoheptanoic acid, 8-aminooctanoic acid, 11-aminoundecanoic acid, 12-aminododecanoic acid, 4-amino-2-hydroxybutyrate, putrescine, cadaverine, hexamethylenediamine, 1,7-diaminoheptane, 1,8-diaminooctane, 1,9-diaminononane, 1,10-diaminodecane, 6-aminohexanol, 7-aminoheptanol, 8-aminooctanol, 9-aminononanol, 10-aminodecanol, 2,4-diaminobutyrate, 2-methylbenzylamine, dihydroxyacetone phosphate, hydroxymethylfurfural, alanine and pyruvate.

In an embodiment, the polypeptide comprises:

i) an amino acid sequence as provided in SEQ ID NO:6,

ii) an amino acid sequence which is at least 40% identical, or at least 80% identical, or at least 90% identical, or at least 95% identical, to SEQ ID NO:6, or

iii) a biologically active fragment of i) or ii).
wherein the polypeptide has amino transferase activity on one, more or all of the substrates selected from, but not limited to, 6-aminohexanoic acid, 7-aminoheptanoic acid, 8-aminooctanoic acid, 11-aminoundecanoic acid, 12-aminododecanoic acid, putrescine, cadaverine, 3-aminocyclohexanoate, propionaldehyde, butyraldehyde, tyramine, 2-aminoindan, 2- methylbenzylamine, hexamethylenediamine, 1,7-diaminoheptane, 1,8-diaminooctane, 1,9-diaminononane, 1,10-diaminodecane, cyclohexylamine, 6-aminohexanol, 7-aminoheptanol, 8-aminooctanol, 9-aminononanol, 10-aminodecanol, cyclohexanone, dopamine, serotonin, alanine and pyruvate.

In an embodiment, the polypeptide comprises:

i) an amino acid sequence as provided in SEQ ID NO:7,

ii) an amino acid sequence which is at least 40% identical, or at least 80% identical, or at least 90% identical, or at least 95% identical, to SEQ ID NO:7, or

iii) a biologically active fragment of i) or ii),
wherein the polypeptide has amino transferase activity on one, more or all of the substrates selected from, but not limited to, glycine, 4-aminobutyrate, 5-aminopentanoate, 6-aminohexanoic acid, 7-aminoheptanoic acid, 8-aminooctanoic acid, 11-aminoundecanoic acid, 12-aminododecanoic acid, ornithine, lysine, 3-aminocyclohexanoate, 4-amino-2-hydroxybutyrate, putrescine, cadaverine, hexamethylenediamine, 1,7-diaminoheptane, 1,8-diaminooctane, 1,9-diaminononane, 1,10-diaminodecane, 6-aminohexanol, 7-aminoheptanol, 8-aminooctanol, 9-aminononanol, 10-aminodecanol, 2,4-diaminobutyrate, 2-methylbenzylamine, dihydroxyacetone phosphate. hydroxymethylfurfural, alanine and pyruvate.

In an embodiment, the polypeptide comprises:

i) an amino acid sequence as provided in SEQ ID NO:8,

ii) an amino acid sequence which is at least 40% identical, or at least 80% identical, or at least 90% identical, or at least 95% identical, to SEQ ID NO:8, or

iii) a biologically active fragment of i) or ii),
wherein the polypeptide has amino transferase activity on one, more or all of the substrates selected from, but not limited to, glycine, 4-aminobutyrate, 5-aminopentanoate, 6-aminohexanoic acid, 7-aminoheptanoic acid, 8-aminooctanoic acid, 11-aminoundecanoic acid, 12-aminododecanoic acid, ornithine, lysine, 3-aminocyclohexanoate, 4-amino-2-hydroxybutyrate, putrescine, cadaverine, diethylaminomalonate, hexamethylenediamine, 1,7-diaminoheptane, 1,8-diaminooctane. 1,9-diaminononane, 1,10-diaminodecane, 6-aminohexanol, 7-aminoheptanol, 8-aminooctanol, 9-aminononanol, 10-aminodecanol, 2,4-diaminobutyrate, 2-methylbenzylamine, dihydroxyacetone phosphate, hydroxymethylfurfural, alanine and pyruvate.

In an embodiment, the polypeptide comprises:

i) an amino acid sequence as provided in SEQ ID NO:9,

ii) an amino acid sequence which is at least 40% identical, or at least 80% identical, or at least 90% identical, or at least 95% identical, to SEQ ID NO:9, or

iii) a biologically active fragment of i) or ii),
wherein the polypeptide has amino transferase activity on one, more or all of the substrates selected from, but not limited to, glycine, 4-aminobutyrate, 5-aminopentanoate, 6-aminohexanoic acid, 7-aminoheptanoic acid, 8-aminooctanoic acid, 11-aminoundecanoic acid, 12-aminododecanoic acid, ornithine, lysine, 3-aminocyclohexanoate, 4-amino-2-hydroxybutyrate, putrescine, cadaverine, N-acetyl-L-ornithine, hexamethylenediamine, 1,7-diaminoheptane, 1,8-diaminooctane, 1,9-diaminononane, 1,10-diaminodecane, 6-aminohexanol, 7-aminoheptanol, 8-aminooctanol, 9-aminononanol, 10-aminodecanol, 2,4-diaminobutyrate, 2-methylbenzylamine, dihydroxyacetone phosphate, hydroxymethylfurfural, alanine and pyruvate.

In an embodiment, the polypeptide comprises:

i) an amino acid sequence as provided in SEQ ID NO:10,

ii) an amino acid sequence which is at least 40% identical, or at least 80% identical, or at least 90% identical, or at least 95% identical, to SEQ ID NO:10, or

iii) a biologically active fragment of i) or ii),
wherein the polypeptide has amino transferase activity on one, more or all of the substrates selected from, but not limited to, glycine, 4-aminobutyrate, 5-aminopentanoate, 6-aminohexanoic acid, 7-aminoheptanoic acid, 8-aminooctanoic acid, 11-aminoundecanoic acid, 12-aminododecanoic acid, ornithine, lysine, 3-aminocyclohexanoate, 4-amino-2-hydroxybutyrate, putrescine, cadaverine, N-acetyl-L-ornithine, hexamethylenediamine, 1,7-diaminoheptane, 1,8-diaminooctane, 1,9-diaminononane, 1,10-diaminodecane, 6-aminohexanol, 7-aminoheptanol, 8-aminooctanol, 9-aminononanol, 10-aminodecanol, 2,4-diaminobutyrate, 2-methylbenzylamine, dihydroxyacetone phosphate, hydroxymethylfurfural, alanine and pyruvate.

In an embodiment, the polypeptide comprises:

i) an amino acid sequence as provided in SEQ ID NO:11,

ii) an amino acid sequence which is at least 40% identical, or at least 80% identical, or at least 90% identical, or at least 95% identical, to SEQ ID NO:11, or

iii) a biologically active fragment of i) or ii).
wherein the polypeptide has amino transferase activity on one, more or all of the substrates selected from, but not limited to, glycine, 4-aminobutyrate, 5-aminopentanoate, 6-aminohexanoic acid, 7-aminoheptanoic acid, 8-aminooctanoic acid, 11-aminoundecanoic acid, 12-aminododecanoic acid, ornithine, lysine, 3-aminocyclohexanoate, 4-amino-2-hydroxybutyrate, putrescine, cadaverine, N-acetyl -L-ornithine, diethylaminomalonate, cyclohexylamine, hexamethylenediamine, 1,7-diaminoheptane, 1,8-diaminooctane, 1,9-diaminononane, 1,10-diaminodecane, 6-aminohexanol, 7-aminoheptanol, 8-aminooctanol, 9-aminononanol, 10-aminodecanol, 2,4-diaminobutyrate, 2-methylbenzylamine, dihydroxyacetone phosphate, hydroxymethylfurfural, alanine and pyruvate.

In an embodiment, the polypeptide comprises:

i) an amino acid sequence as provided in SEQ ID NO:12.

ii) an amino acid sequence which is at least 40% identical, or at least 80% identical, or at least 90% identical, or at least 95% identical, to SEQ ID NO:12, or

iii) a biologically active fragment of i) or ii),
wherein the polypeptide has amino transferase activity on one, more or all of the substrates selected from, but not limited to, glycine, 4-aminobutyrate, 5-aminopentanoate, 6-aminohexanoic acid, 7-aminoheptanoic acid, 8-aminooctanoic acid, 11-aminoundecanoic acid, 12-aminododecanoic acid, ornithine, lysine, 3-aminocyclohexanoate, 4-amino-2-hydroxybutyrate, putrescine, cadaverine, N-acetyl-L-ornithine, diethylaminomalonate, cyclohexylamine, hexamethylenediamine, 1,7-diaminoheptane, 1,8-diaminooctane, 1,9-diaminononane, 1,10-diaminodecane, 6-aminohexanol, 7-aminoheptanol, 8-aminooctanol, 9-aminononanol, 10-aminodecanol, 2,4-diaminobutyrate, 2-methylbenzylamine, dihydroxyacetone phosphate, hydroxymethylfurfural, alanine and pyruvate.

The polypeptides of the invention can be used to produce an industrial product.

As used herein, the term “industrial product” refers to a product that is manufactured on a commercial scale for human use. The industrial product may be an intermediate product that can be sold or used for production of a further product. For example, the polypeptides of the present invention may be used to produce amines which are themselves considered to be industrial products. These amines could be sold and used as building blocks for the synthesis of polyamides such as nylons (i.e., a further industrial product). The industrial product may be a mixture of products, for example, a mixture of amino acids and amines The industrial product may be further reacted with, for example, one or more enzymes or compounds or itself, to produce another industrial product. A skilled person will appreciate that the industrial product may be a monomer that can be reacted with itself or other monomers to form a polymer.

### Polynucleotides

The present invention refers to various polynucleotides.

The terms “polynucleotide”, and “nucleic acid” are used interchangeably. A polynucleotide is a polymer of nucleotide monomers. A polynucleotide of the invention may be of any length and can comprise deoxyribonucleotides or ribonucleotides, or analogs thereof, or a mixture thereof. A polynucleotide of the invention may be of genomic, cDNA, semisynthetic, or synthetic origin, double-stranded or single-stranded and by virtue of its origin or manipulation: (1) is not associated with all or a portion of a polynucleotide with which it is associated in nature, (2) is linked to a polynucleotide other than that to which it is linked in nature (for example, a promoter), or (3) does not occur in nature. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), ribozymes, cDNA, recombinant polynucleotides, branched polynucleotides, plasmids, vectors, isolated DNA, isolated RNA, chimeric DNA, nucleic acid probes, and primers. A polynucleotide may comprise modified nucleotides such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization such as by conjugation with a labeling component.

By “isolated polynucleotide” it is meant a polynucleotide which has generally been separated from the polynucleotide sequences with which it is associated or linked in its native state. Preferably, the isolated polynucleotide is at least 60% free, more preferably at least 75% free, and more preferably at least 90% free from the polynucleotide sequences with which it is naturally associated or linked.

By “exogenous polynucleotide” it is meant a polynucleotide present in a cell free expression system or a cell that does not naturally comprise the polynucleotide or a polynucleotide expressed in an altered amount or expressed at an altered rate (e.g., in the case of mRNA) compared to its native state. In an embodiment, the polynucleotide is introduced into a cell that does not naturally comprise the polynucleotide. Typically an exogenous DNA is used as a template for transcription of mRNA which is then translated into a continuous sequence of amino acid residues coding for a polypeptide of the invention within the transformed cell. In another embodiment, the polynucleotide is endogenous to the cell and its expression is altered by recombinant means, for example, an exogenous control sequence is introduced upstream of an endogenous gene of interest to enable the transformed cell to express the polypeptide encoded by the gene.

An exogenous polynucleotide of the invention includes polynucleotides which have not been separated from other components of the cell-based or cell-free expression system, in which it is present, and polynucleotides produced in said cell-based or cell-free systems which are subsequently purified away from at least some other components.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 50%, more preferably at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

A polynucleotide of, or useful for, the present invention may selectively hybridise, under stringent conditions, to a polynucleotide defined herein. As used herein, stringent conditions are those that: (1) employ during hybridisation a denaturing agent such as formamide, for example, 50% (v/v) formamide with 0.1% (w/v) bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (2) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS and 10% dextran sulfate at 42° C. in 0.2×SSC and 0.1% SDS, and/or (3) employ low ionic strength and high temperature for washing, for example, 0.015 M NaCl/0.0015 M sodium citrate/0.1% SDS at 50° C.

Polynucleotides of the invention may possess, when compared to reference polynucleotides, one or more mutations which are deletions, insertions, or substitutions of nucleotide residues. Polynucleotides which have mutations relative to a reference sequence can be either naturally occurring (that is to say, isolated from a natural source) or synthetic (for example, by performing site-directed mutagenesis or DNA shuffling on the nucleic acid).

### Recombinant Vectors

One embodiment of the present invention includes a recombinant vector, which comprises at least one polynucleotide defined herein and is capable of delivering the polynucleotide into a host cell. Recombinant vectors include expression vectors. Recombinant vectors contain heterologous polynucleotide sequences, i.e., polynucleotide sequences that are not naturally found adjacent to polynucleotides of the present invention, that are preferably derived from a species other than the species from which the polynucleotides of the present invention are derived. The vector can be either RNA or DNA, either prokaryotic or eukaryotic, and typically is a viral vector, derived from a virus, or a plasmid.

Plasmid vectors typically include additional nucleic acid sequences that provide for easy selection, amplification, and transformation in prokaryotic cells, for example, pUC-derived vectors, pSK-derived vectors, pGEM-derived vectors, pSP-derived vectors, pBS-derived vectors, or binary vectors containing one or more T-DNA regions. Additional nucleic acid sequences include origins of replication to provide for autonomous replication of the vector, selectable marker genes, preferably encoding antibiotic or herbicide resistance, unique multiple cloning sites providing for multiple sites to insert nucleic acid sequences or genes encoded in the nucleic acid construct, and sequences that enhance transformation of prokaryotic and eukaryotic cells.

“Operably linked” as used herein, refers to a functional relationship between two or more nucleic acid (e.g., DNA) segments. Typically, it refers to the functional relationship of transcriptional regulatory element (promoter) to a transcribed sequence. For example, a promoter is operably linked to a coding sequence of a polynucleotide defined herein, if it stimulates or modulates the transcription of the coding sequence in an appropriate cell. Generally, promoter transcriptional regulatory elements that are operably linked to a transcribed sequence are physically contiguous to the transcribed sequence, i.e., they are cis-acting. However, some transcriptional regulatory elements such as enhancers. need not be physically contiguous or located in close proximity to the coding sequences whose transcription they enhance.

When there are multiple promoters present, each promoter may independently be the same or different.

Recombinant vectors may also contain: (a) one or more secretory signals which encode signal peptide sequences, to enable an expressed polypeptide defined herein to be secreted from the cell that produces the polypeptide, or which provide for localisation of the expressed polypeptide, for example, for retention of the polypeptide in the endoplasmic reticulum (ER) in the cell, or transfer into a plastid, and/or (b) contain fusion sequences which lead to the expression of nucleic acid molecules as fusion proteins. Examples of suitable signal segments include any signal segment capable of directing the secretion or localisation of a polypeptide defined herein. Recombinant vectors may also include intervening and/or untranslated sequences surrounding and/or within the nucleic acid sequence of a polynucleotide defined herein.

To facilitate identification of transformants, the recombinant vector desirably comprises a selectable or screenable marker gene as, or in addition to, the nucleic acid sequence of a polynucleotide defined herein. By “marker gene” is meant a gene that imparts a distinct phenotype to cells expressing the marker gene and thus, allows such transformed cells to be distinguished from cells that do not have the marker. A selectable marker gene confers a trait for which one can “select” based on resistance to a selective agent (e.g., a herbicide, antibiotic, radiation, heat, or other treatment damaging to untransformed cells). A screenable marker gene (or reporter gene) confers a trait that one can identify through observation or testing, i.e., by “screening” (e.g., (3-glucuronidase, luciferase, GFP or other enzyme activity not present in untransformed cells). The marker gene and the nucleotide sequence of interest do not have to be linked—co-transformation of unlinked genes is described in, for example, U.S. Pat. No. 4,399,216. The actual choice of a marker is not crucial as long as it is functional (i.e., selective) in combination with the host cell.

Examples of bacterial selectable markers are markers that confer antibiotic resistance such as ampicillin, erythromycin, chloramphenicol, or tetracycline resistance, preferably kanamycin resistance. Exemplary selectable markers for selection of plant transformants include, but are not limited to, a hyg gene which encodes hygromycin B resistance; a neomycin phosphotransferase (nptII) gene conferring resistance to kanamycin, paromomycin, G418; a glutathione-S-transferase gene from rat liver conferring resistance to glutathione derived herbicides as, for example, described in EP 256223; a glutamine synthetase gene conferring, upon overexpression, resistance to glutamine synthetase inhibitors such as phosphinothricin as, for example, described in WO 87/05327; an acetyltransferase gene from Streptomyces viridochromogenes conferring resistance to the selective agent phosphinothricin as, for example, described in EP 275957; a gene encoding a 5-enolshikimate-3-phosphate synthase (EPSPS) conferring tolerance to N-phosphonomethylglycine as, for example, described by Hinchee et al. (1988); a bar gene conferring resistance against bialaphos as, for example, described in WO 91/02071; a nitrilase gene such as b×n from Klebsiella ozaenae which confers resistance to bromoxynil (Stalker et al., 1988); a dihydrofolate reductase (DHFR) gene conferring resistance to methotrexate (Thillet et al., 1988); a mutant acetolactate synthase gene (ALS) which confers resistance to imidazolinone, sulfonylurea, or other ALS-inhibiting chemicals (EP 154,204); a mutated anthranilate synthase gene that confers resistance to 5-methyl tryptophan; or a dalapon dehalogenase gene that confers resistance to the herbicide.

Preferred screenable markers include, but are not limited to, a uidA gene encoding a β-glucuronidase (GUS) enzyme for which various chromogenic substrates are known; a β-galactosidase gene encoding an enzyme for which chromogenic substrates are known; an aequorin gene (Prasher et al., 1985) which may be employed in calcium-sensitive bioluminescence detection; a green fluorescent protein gene (Niedz et al., 1995) or derivatives thereof; or a luciferase (luc) gene (Ow et al., 1986) which allows for bioluminescence detection. By “reporter molecule” it is meant a molecule that, by its chemical nature, provides an analytically identifiable signal that facilitates determination of promoter activity by reference to protein product.

Preferably, the recombinant vector is stably incorporated into the genome of the cell. Accordingly, the recombinant vector may comprise appropriate elements which allow the vector to be incorporated into the genome, or into a chromosome of the cell.

### Expression Vector

As used herein, an “expression vector” is a DNA or RNA vector that is capable of transforming a host cell and of effecting expression of one or more specified polynucleotides. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in host cells of the present invention, including in bacterial, fungal, endoparasite, arthropod, animal, plant and algal cells. Particularly preferred expression vectors of the present invention can direct gene expression in bacterial or fungal cells.

Expression vectors of the present invention contain regulatory sequences such as transcription control sequences, translation control sequences, origins of replication, and other regulatory sequences that are compatible with the host cell and that control the expression of polynucleotides of the present invention. In particular, expression vectors of the present invention include transcription control sequences. Transcription control sequences are sequences which control the initiation, elongation, and termination of transcription. Particularly important transcription control sequences are those which control transcription initiation such as promoter, enhancer, operator and repressor sequences. Suitable transcription control sequences include any transcription control sequence that can function in a host cell of the present invention. The choice of the regulatory sequences used depends on the host cell. Such regulatory sequences may be obtained from any eukaryotic organism, or may be chemically synthesized. A variety of such transcription control sequences are known to those skilled in the art.

Preferred transcription control sequences include those which function in bacterial, fungal, arthropod, nematode, plant or mammalian cells, such as, but not limited to, tac, lac, trp, trc, oxy-pro, omp/lpp, rrnB, bacteriophage lambda, bacteriophage T7, T7lac, bacteriophage T3, bacteriophage SP6, bacteriophage SP01, metallothionein, alpha-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpesvirus, raccoon poxvirus, other poxvirus, adenovirus, cytomegalovirus (such as intermediate early promoters), simian virus 40, retrovirus, actin, retroviral long terminal repeat, Rous sarcoma virus, heat shock, phosphate and nitrate transcription control sequences, as well as other sequences capable of controlling gene expression in prokaryotic or eukaryotic cells.

The 5′ non-translated leader sequence can be derived from the promoter selected to express the heterologous gene sequence of the polynucleotide of the present invention, or may be heterologous with respect to the coding region of the enzyme to be produced, and can be specifically modified if desired so as to increase translation of mRNA. For a review of optimizing expression of transgenes, see Koziel et al. (1996). The present invention is not limited to constructs wherein the non-translated region is derived from the 5′ non-translated sequence that accompanies the promoter sequence. The leader sequence could also be derived from an unrelated promoter or coding sequence.

The termination of transcription is accomplished by a 3′ non-translated DNA sequence operably linked in the expression vector to the polynucleotide of interest. The 3′ non-translated region of a recombinant DNA molecule contains a polyadenylation signal that functions in the host cell to cause the addition of adenylate nucleotides to the 3′ end of the RNA.

Recombinant DNA technologies can be used to improve expression of a transformed polynucleotide by manipulating for example, the number of copies of the polynucleotide within a host cell, the efficiency with which those polynucleotide are transcribed, the efficiency with which the resultant transcripts are translated, and the efficiency of post-translational modifications. Recombinant techniques useful for increasing the expression of polynucleotides defined herein include, but are not limited to, operatively linking the polynucleotide to a high-copy number plasmid, integration of the polynucleotide molecule into one or more host cell chromosomes, addition of vector stability sequences to the plasmid, substitutions or modifications of transcription control signals (e.g., promoters, operators, enhancers), substitutions or modifications of translational control signals (e.g., ribosome binding sites, Shine-Dalgarno sequences), modification of the polynucleotide to correspond to the codon usage of the host cell, and the deletion of sequences that destabilize transcripts.

### Host and Recombinant Cells

As used herein, the term “host cell” refers to a cell capable of being transformed with an exogenous polynucleotide of the invention. Once transformed, the host cell can be referred to as a “recombinant cell” or “transgenic cell”.

The term “recombinant cell” includes direct or indirect progeny cells thereof comprising the polynucleotide.

Transformation of a polynucleotide into a host cell can be accomplished by any method by which a polynucleotide can be inserted into the cell. Transformation techniques include, but are not limited to, transfection, electroporation, microinjection, lipofection, adsorption, and protoplast fusion.

A recombinant cell may remain unicellular or may grow into a tissue, organ or a multicellular organism. Transformed polynucleotides of the present invention can remain extrachromosomal or can integrate into one or more sites within a chromosome of the transformed (i.e., recombinant) cell in such a manner that their ability to be expressed is retained.

The recombinant cell may be a cell in culture, a cell in vitro, or in an organism or part thereof. In one embodiment, the recombinant cell is a non-human cell.

Suitable host cells to transform include any cell that can be transformed with a polynucleotide of the present invention. Host cells of the present invention can either be endogenously (i.e.. naturally) capable of producing polypeptides of the present invention or can be capable of producing such polypeptides after being transformed with at least one polynucleotide of the present invention. Host cells of the present invention can be any cell capable of producing at least one protein of the present invention, and include bacterial, fungal (including yeast such as Candida sp. and Saccharomyces), filamentous fungal cells (such as Penicillium and Aspergillus), parasite, nematode, arthropod, animal and plant cells. Examples of host cells include Salmonella, Escherichia, Bacillus, Listeria, Pseudomonas, Saccharomyces, Spodoptera, Mycobacteria, Trichoplusia, BHK (baby hamster kidney) cells, MDCK cells, CRFK cells, CV-1 cells, COS (e.g., COS-7) cells, and Vero cells. Further examples of host cells are E. coli, including E. coli K-12 derivatives; Salmonella typhi; Salmonella typhimurium, including attenuated strains; Spodoptera frugiperda; Trichoplusiani; and non-tumorigenic mouse myoblast G8 cells (e.g.. ATCC CRL 1246).

Particularly preferred host cells are E. coli, Pseudomonas, Bacillus, Candida sp., Saccharomyces, Penicillium and Aspergillus.

### Transgenic Non-Human Organism

The term “transgenic non-human organism” refers to, for example, a non-human animal, plant, or a fungus comprising an exogenous polynucleotide (transgene) or a recombinant polypeptide of the invention.

A “transgene” is a gene that has been introduced into the genome by a transformation procedure. The term includes a gene in a cell or non-human organism or part thereof which was introduced into the genome of a progenitor cell thereof. Progeny of a cell or non-human organism may be at least a 3^{rd} or 4^{th} generation of the progenitor cell or non-human organism. Progeny may be produced by sexual reproduction or vegetatively such as, for example, from tubers in potatoes or ratoons in sugarcane. The term “genetically modified”, and variations thereof, is a broader term that includes introducing a gene into a cell by transformation or transduction, mutating a gene in a cell and genetically altering or modulating the regulation of a gene in a cell, or the progeny of any cell modified as described above.

As used herein, the term “wild-type” or variations thereof refers to a cell, or non-human organism or part thereof that has not been genetically modified.

Transgenic Plants

The term “plant” as used herein refers to whole plants, such as, for example, a plant growing in a field, and any substance which is present in, obtained from, derived from, or related to a plant, such as, for example, vegetative structures (e.g., leaves, stems), roots, floral organs/structures. seeds (including embryo, endosperm, and seed coat), plant tissue (e.g., vascular, tissue, ground tissue, and the like), cells (e.g., pollen), and progeny thereof.

Plants contemplated for use in the practice of the present invention include both monocotyledons and dicotyledons. Target plants include, but are not limited to, the following: cereals (wheat, barley, rye, oats, rice, sorghum, triticale, and related crops); beet (sugar beet and fodder beet); ponies (apples, pears), stone fruit (plums, peaches, almonds, cherries), tropical fruit (bananas, pineapple, pawpaws) and soft fruit (cherries, strawberries, raspberries and black-berries); leguminous plants (beans, lentils, peas, soybeans, lucerne, lupins); oil plants (rape, mustard, poppy, olives, sunflowers, coconut, castor oil plants, cocoa beans, groundnuts); cucumber plants (marrows, cucumbers, melons); fibre plants (cotton, cotton defoliant, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocados, cinnamon, camphor); or plants such as maize, tobacco, nuts, coffee, sugar cane, tea, vines, hops, turf including perennial grass and the phalarsis cultivars sirolan and sirone, and natural rubber plants, as well as ornamentals (flowers such as daffodils, gladioli and tulips, shrubs such as Duboisia, broad-leaved trees and evergreens, such as conifers). Preferably, the plants are angiosperms.

Transgenic plants, as defined in the context of the present invention include plants (as well as parts and cells of said plants) and their progeny which have been genetically modified using recombinant techniques to cause production of at least one polypeptide of the present invention in the desired plant or plant organ. Transgenic plants can be produced using techniques known in the art, such as those generally described in A. Slater et al., Plant Biotechnology—The Genetic Manipulation of Plants, Oxford University Press (2003); and P. Christou and H. Klee, Handbook of Plant Biotechnology, John Wiley and Sons (2004).

In a preferred embodiment of the invention, the transgenic plants are homozygous for each and every gene that has been introduced (transgene) so that their progeny do not segregate for the desired phenotype. The transgenic plants may also be heterozygous for the introduced transgene(s), such as, for example, in F1 progeny which have been grown from hybrid seed. Such plants may provide advantages such as hybrid vigour, well known in the art.

A polynucleotide of the present invention may be expressed constitutively in the transgenic plants during all stages of development. Depending on the use of the plant or plant organs, the polypeptides may be expressed in a stage-specific manner. Furthermore, the polynucleotides may be expressed tissue-specifically.

Regulatory sequences which are known or are found to cause expression of a gene encoding a polypeptide of interest in plants may be used in the present invention. The choice of the regulatory sequences used depends on the target plant and/or target organ of interest. Such regulatory sequences may be obtained from plants or plant viruses, or may be chemically synthesized. Such regulatory sequences are well known to those skilled in the art.

A number of vectors suitable for stable transfection of plant cells or for the establishment of transgenic plants have been described inp for examplep Pouwels et al., Cloning Vectors: A Laboratory Manual (1985, supp. 1987); Weissbach and Weissbach, Methods for Plant Molecular Biology. Academic Press (1989); and Gelvin et al., Plant Molecular Biology Manual, Kluwer Academic Publishers (1990). Typically, plant expression vectors include, for example, one or more cloned plant genes under the transcriptional control of 5′ and 3′ regulatory sequences and a dominant selectable marker. Such plant expression vectors can also contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

A number of constitutive promoters that are active in plant cells have been described. Suitable promoters for constitutive expression in plants include, but are not limited to, the cauliflower mosaic virus (CaMV) 35S promoter, the Figwort mosaic virus (FMV) 35S, the sugarcane bacilliform virus promoter, the commelina yellow mottle virus promoter, the light-inducible promoter from the small subunit of the ribulose-1,5-bis-phosphate carboxylase, the rice cytosolic triosephosphate isomerase promoter, the adenine phosphoribosyltransferase promoter of Arabidopsis, the rice actin 1 gene promoter, the mannopine synthase and octopine synthase promoters, the Adh promoter, the sucrose synthase promoter, the R gene complex promoter, and the chlorophyll α/β binding protein gene promoter. These promoters have been used to create DNA vectors that have been expressed in plants; see for example, PCT publication WO 84/02913. All of these promoters have been used to create various types of plant-expressible recombinant DNA vectors.

The 5′ non-translated leader sequence can be derived from the promoter selected to express the heterologous gene sequence of the polynucleotide of the present invention, and can be specifically modified if desired so as to increase translation of mRNA. For a review of optimizing expression of transgenes, see Koziel et al. (1996). The 5′ non-translated regions can also be obtained from plant viral RNAs (tobacco mosaic virus, tobacco etch virus, maize dwarf mosaic virus, alfalfa mosaic virus, among others) from suitable eukaryotic genes, plant genes (wheat and maize chlorophyll a/b binding protein gene leader), or from a synthetic gene sequence. The present invention is not limited to constructs wherein the non-translated region is derived from the 5′ non-translated sequence that accompanies the promoter sequence. The leader sequence could also be derived from an unrelated promoter or coding sequence. Leader sequences useful in context of the present invention comprise the maize Hsp70 leader (see U.S. Pat. No. 5,362,865 and U.S. Pat. No. 5,859,347), and the TMV omega element.

The termination of transcription is accomplished by a 3′ non-translated DNA sequence operably linked in the chimeric vector to the polynucleotide of interest. The 3′ non-translated region of a recombinant DNA molecule contains a polyadenylation signal that functions in plants to cause the addition of adenylate nucleotides to the 3′ end of the RNA. The 3′ non-translated region can be obtained from various genes that are expressed in plant cells. The nopaline synthase 3′ untranslated region, the 3′ untranslated region from pea small subunit Rubisco gene, the 3′ untranslated region from soybean 7S seed storage protein gene are commonly used in this capacity. The 3′ transcribed, non-translated regions containing the polyadenylate signal of Agrobacterium tumor-inducing (Ti) plasmid genes are also suitable.

Four general methods for direct delivery of a gene into cells have been described: (1) chemical methods (Graham et at, 1973); (2) physical methods such as microinjection (Capecchi, 1980); electroporation (see WO 87/06614, U.S. Pat. Nos. 5,472,869, 5,384,253. WO 92/09696 and WO 93/21335); and the gene gun (see U.S. Pat. Nos. 4,945,050 and U.S. Pat. No. 5,141,131); (3) viral vectors (Clapp, 1993; Lu et al., 1993; Eglitis et al., 1988); and (4) receptor-mediated mechanisms (Curiel et al., 1992; Wagner et al., 1992).

To confirm the presence of the transgenes in transgenic cells and plants, a polymerase chain reaction (PCR) amplification or Southern blot analysis can be performed using methods known to those skilled in the art. Expression products of the transgenes can be detected in any of a variety of ways, depending upon the nature of the product, and include Western blot and enzyme assay. One particularly useful way to quantitate protein expression and to detect replication in different plant tissues is to use a reporter gene, such as GUS. Once transgenic plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics.

Transgenic Non-Human Animals

A “transgenic non-human animal” refers to an animal, other than a human, that contains a gene construct (“transgene”) not found in a wild-type animal of the same species or breed. Techniques for producing transgenic animals are well known in the art. A useful general textbook on this subject is Houdebine, Transgenic animals—Generation and Use, Harwood Academic (1997).

Heterologous DNA can be introduced, for example, into fertilized mammalian ova. For instance, totipotent or pluripotent stem cells can be transformed by microinjection, calcium phosphate mediated precipitation, liposome fusion, retroviral infection or other means. The transformed cells are then introduced into the embryo, and the embryo then develops into a transgenic animal. In a highly preferred method, developing embryos are infected with a retrovirus containing the desired DNA, and transgenic animals produced from the infected embryo. In a most preferred method, however, the appropriate DNAs are coinjected into the pronucleus or cytoplasm of embryos, preferably at the single cell stage, and the embryos allowed to develop into mature transgenic animals.

Another method used to produce a transgenic animal involves microinjecting a nucleic acid into pro-nuclear stage eggs by standard methods. Injected eggs are then cultured before transfer into the oviducts of pseudopregnant recipients.

Transgenic animals may also be produced by nuclear transfer technology. Using this method, fibroblasts from donor animals are stably transfected with a plasmid incorporating the coding sequences for a binding domain or binding partner of interest under the control of regulatory sequences. Stable transfectants are then fused to enucleated oocytes, cultured and transferred into female recipients.

### Compositions

Compositions of the present invention can include a carrier. The carrier may be solid or liquid. Useful examples of carriers include, but are not limited to, diluents, solvents, surfactants, excipients, suspending agents, buffering agents, lubricating agents, adjuvants, vehicles, emulsifiers, absorbants, dispersion media, coatings, stabilizers, protective colloids, adhesives, thickeners, thixotropic agents, penetration agents, sequestering agents, isotonic and absorption delaying agents that do not affect the activity of the active agents of the disclosure.

Examples of excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability.

Examples of buffers include phosphate buffer, bicarbonate buffer and Tris buffer, while examples of preservatives include thimerosal or o-cresol, formalin and benzyl alcohol. Excipients can also be used to increase the half-life of a composition, for example, but are not limited to, polymeric controlled release vehicles, biodegradable implants, liposomes, bacteria, viruses, other cells, oils, esters, and glycols.

Furthermore, a polypeptide described herein can be provided in a composition that enhances the rate and/or degree of amino transferase activity, or increases the stability of the polypeptide. For example, the polypeptide can be immobilized on a polyurethane matrix (Gordon et al., 1999), or encapsulated in appropriate liposomes (Petrikovics et al., 2000a and b). The polypeptide can also be incorporated into a composition comprising a foam, such as those used routinely in fire-fighting (LeJeune et al., 1998).

One embodiment of the present invention is a controlled release formulation that is capable of slowly releasing a composition of the present invention. As used herein, a “controlled release formulation” comprises a composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Preferred controlled release formulations are biodegradable (i.e., bioerodible).

The concentration of the polypeptide, vector, bacteria, extract, or host cell etc., of the present invention that will be required to produce effective compositions for catalysing transfer of an amino group from an amino donor to an amino acceptor will depend on the nature of the substrate, the concentration of the substrate, and the formulation of the composition. The effective concentration of the polypeptide, vector, bacteria, extract, or host cell etc., within the composition can readily be determined experimentally, as will be understood by the skilled person.

As used herein, the term “extract” refers to a composition comprising one or more components obtained from a cell, cell free system, culture medium, or non-human organism of the invention. The extract comprises a polypeptide of the invention. The term “extract” is also intended to cover supernatant that comprises a secreted form of a polypeptide of the invention.

### Antibodies

The term “antibody” as used in this invention includes polyclonal antibodies, monoclonal antibodies, bispecific antibodies, diabodies, triabodies, heteroconjugate antibodies, chimeric antibodies including intact molecules as well as fragments thereof, such as Fab, F(ab′)₂, and Fv which are capable of binding the epitopic determinant, and other antibody-like molecules.

Antibody fragments retain some ability to selectively bind with its antigen and are defined as follows:

(1) Fab. the fragment which contains a monovalent antigen-binding fragment of an antibody molecule can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (“SCA”), defined as a genetically engineered molecule containing the variable region of the light chain, the variable region of the heavy chain, linked by a suitable polypeptide linker as a genetically fused single chain molecule.

Methods of making these fragments are known in the art (see for example, Harlow and Lane, Antibodies: A Laboratory Manual, Cold Spring Harbor Laboratory, New York (1988)).

(6) Single domain antibody, typically a variable heavy domain devoid of a light chain.

The term “specifically binds” refers to the ability of the antibody to bind to at least one polypeptide of the present invention but not other known proteins.

As used herein, the term “epitope” refers to a region of a polypeptide of the invention which is bound by the antibody. An epitope can be administered to an animal to generate antibodies against the epitope, however, antibodies of the present invention preferably specifically bind the epitope region in the context of the entire polypeptide.

If polyclonal antibodies are desired, a selected mammal (e.g., mouse, rabbit, goat, horse, etc.) is immunised with an immunogenic polypeptide of the invention. Serum from the immunised animal is collected and treated according to known procedures. If serum containing polyclonal antibodies contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals.

Monoclonal antibodies directed against polypeptides of the invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

Other techniques for producing antibodies of the invention are known in the art.

Antibodies of the invention may be bound to a solid support and/or packaged into kits in a suitable container along with suitable reagents, controls, instructions and the like.

In an embodiment, antibodies of the present invention are detectably labeled. Exemplary detectable labels that allow for direct measurement of antibody binding include radiolabels, fluorophores, dyes, magnetic beads, chemiluminescers, colloidal particles, and the like. Examples of labels which permit indirect measurement of binding include enzymes where the substrate may provide for a coloured or fluorescent product. Additional exemplary detectable labels include covalently bound enzymes capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. Further, exemplary detectable labels include biotin, which binds with high affinity to avidin or streptavidin; fluorochromes (e.g., phycobiliproteins, phycoerythrin and allophycocyanins; fluorescein and Texas red), which can be used with a fluorescence activated cell sorter; haptens; and the like. Preferably, the detectable label allows for direct measurement in a plate luminometer, for example, biotin. Such labeled antibodies can be used in techniques known in the art to detect polypeptides of the invention.

### Identification of Amino Transferases and Compounds that Bind Thereto

Enhanced Amino Transferase Activity or Altered Substrate Specificity

In one aspect, the invention provides a method for identifying an acyltransferase having enhanced ability to catalyse the transfer of an amino group from an amino acid or an amine compound to an amino acceptor, or altered substrate specificity.

The method comprises altering one or more amino acids of a polypeptide of the invention. Mutants may be engineered using standard procedures in the art (see above) such as by introducing appropriate nucleotide changes into a nucleic acid defined herein, or by in vitro synthesis of the desired polypeptide. Mutant polypeptides can readily be screened using techniques described herein to determine if they possess amino transferase activity, for example, using a coupled dehydrogenase assay (Bernt and Bergmeyer, 1974).

For example, a polynucleotide comprising a sequence as shown in any one or more of SEQ ID NOS:3 to 5, or 14 to 20 which encodes an amino transferase may be mutated and/or recombined at random to create a large library of gene variants (mutants) using for example, error-prone PCR and/or DNA shuffling. Mutants may be selected for further investigation on the basis that they comprise a conserved amino acid motif.

Direct PCR sequencing of the nucleic acid or a fragment thereof may be used to determine the exact nucleotide sequence and deduce the corresponding amino acid sequence and thereby identify conserved amino acid sequences. Degenerate primers based on conserved amino acid sequences may be used to direct PCR amplification. Degenerate primers can also be used as probes in DNA hybridization assays. Alternatively, the conserved amino acid sequence(s) may be detected in protein hybridization assays that utilize for example, an antibody that specifically binds to the conserved amino acid sequences(s), or a substrate that specifically binds to the conserved amino acid sequences(s).

A cell comprising a nucleic acid molecule encoding an amino transferase operably linked to a promoter which is active in the cell may be obtained using standard procedures in the art such as by introducing the nucleic acid molecule into a cell by, for example, calcium phosphate precipitation, polyethylene glycol treatment, electroporation, and combinations of these treatments. Other methods of cell transformation can also be used and include, but are not limited to, the introduction of DNA into plants by direct DNA transfer or injection. Transformed plant cells may also be obtained using Agrobacterium-mediated transfer and acceleration methods as described herein.

The method further comprises determining if the amino transferase activity is increased when compared to the parent unaltered polypeptide using known techniques in the art. For example, using the coupled assays described previously, HPLC, NMR, or mass spectrometry.

As used herein “compared with” refers to comparing levels of the amino transferase activity of an altered polypeptide or a cell or a transgenic non-human organism expressing the altered polypeptide with a polypeptide, cell or transgenic non-human organism of the invention.

Three Dimensional Structure of a Polypeptide of the Invention

As used herein, the term “crystal” means a structure (such as a three dimensional (3D) solid aggregate) in which the plane faces intersect at definite angles and in which there is a regular structure (such as internal structure) of the constituent chemical species. The term “crystal” refers in particular to a solid physical crystal form such as an experimentally prepared crystal.

It will be understood that any reference herein to the atomic coordinates or subset of the atomic coordinates shown in Appendix I shall include, unless specified otherwise, atomic coordinates having a root mean square deviation of backbone atoms of not more than 1.5 Å, preferably not more than 1 Å, when superimposed on the corresponding backbone atoms described by the atomic coordinates shown in Appendix I. The following defines what is intended by the term “root mean square deviation (RMSD)” between two data sets. For each element in the first data set, its deviation from the corresponding item in the second data set is computed. The squared deviation is the square of that deviation, and the mean squared deviation is the mean of all these squared deviations. The root mean square deviation is the square root of the mean squared deviation. Preferred variants are those in which the RMSD of the x, y and z coordinates for all backbone atoms other than hydrogen is less than 1.5 Å (preferably less than 1 Å, 0.7 Å or less than 0.3 Å) compared with the coordinates given in

Appendix I. It will be readily appreciated by those skilled in the art that a 3D rigid body rotation and/or translation of the atomic coordinates does not alter the structure of the molecule concerned.

The three-dimensional structure of a polypeptide of the invention, such as SEQ ID NO:1 or SEQ ID NO:2, can be used in a method of the invention such as a computer-assisted method of identifying a compound (for example, a substrate, a co-factor, an antagonist or an agonist) that binds a polypeptide as defined herein.

For example, substrates, co-factors, antagonists or agonists can be identified through the use of computer modeling using a docking program such as GRAM, DOCK, or AUTODOCK (Dunbrack et al., 1997). Computer programs can also be employed to estimate the attraction, repulsion, and steric hindrance of a candidate compound to the polypeptide. For example, when trying to identify a substrate (i.e, an amino donor or amino acceptor) of a polypeptide defined herein, docking studies can be used to assess the level of steric interference in the binding pocket(s) of the enzyme. Generally the tighter the fit (e.g., the lower the steric hindrance, and/or the greater the attractive force) the more likely the compound is a substrate.

The three-dimensional structure of a polypeptide of the invention, such as SEQ ID NO:1 or SEQ ID NO:2, can also be used to identify other amino transferases. For example, homology modelling, also known as comparative modelling of a protein, can be used to construct a three-dimensional structural model of a candidate polypeptide from its amino acid sequence (query sequence). This model can used to assess the likelihood that a polypeptide sequence encodes an amino transferase. The three-dimensional structure of a polypeptide of the invention, such as SEQ ID NO:1 or SEQ ID NO:2, can also be used to develop an in silico method for sequence based prediction of substrate specificity. Identification of key amino acids residues responsible for the desired activity can be followed by exploration of public databases to identify candidate enzymes carrying the desired amino acid substitutions.

The three-dimensional structure of a polypeptide of the invention, such as SEQ ID NO:1 or SEQ ID NO:2, can also be used to redesign substrate specificity of the transferase. Binding pockets of the transferase can be re-designed by using a combination of in silico modelling, site-saturated mutagenesis and directed evolution.

For most types of models, standard molecular force fields, representing the forces between constituent atoms and groups, are necessary, and can be selected from force fields known in physical chemistry. Exemplary force fields that are known in the art and can be used in such methods include, but are not limited to, the Constant Valence Force Field (CVFF), the AMBER force field and the CHARM force field. The incomplete or less accurate experimental structures can serve as constraints on the complete and more accurate structures computed by these modeling methods.

Further examples of molecular modeling systems are the CHARMm and QUANTA programs (Polygen Corporation, Waltham, Mass.). CHARMm performs the energy minimization and molecular dynamics functions. QUANTA performs the construction, graphic modeling and analysis of molecular structure. QUANTA allows interactive construction, modification, visualization, and analysis of the behaviour of molecules with each other.

As used herein, a “subset” of the atomic coordinates provided in Appendix I refers to a group of the co-ordinates which can be used in a method of the invention such as a computer-assisted method of identifying a compound (for example, a substrate, a co-factor, an antagonist or an agonist) that binds p6 or a polypeptide defined herein, or a computer-assisted method for identifying other amino transferases or for redesigning substrate specificity of the transferase.

### EXAMPLES

Background

The long chain amino acids (e.g., C9-12) that are needed for nylon manufacture are not abundant in nature, with no clear physiological role for these molecules. Literature searches to identify biocatalysts for these substrates were also unsuccessful. Of relevance in this area, however, was the ability of omega-transaminases such as the well characterised GabT (Gamma-aminobutyrate Transaminase; E.C. 2.6.1.19) to produce short chain omega-amino acids (C4-C7 reported) from aliphatic carboxylic acid semialdehydes as shown below.

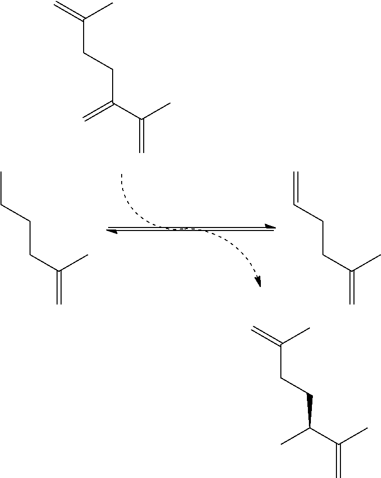

Gamma-aminobutyrate, the substrate for GabT, is an important neurotransmitter in the mammalian central nervous system, and as a result, both the molecule and protein have been the subjects of extensive medical research, affording a wealth of information in the area, but leaving biocatalytic potential unexplored.

DNA encoding the GabT protein from E. coli strain BL21-DE3 was amplified from genomic DNA using specially designed primers. The PCR product was restricted using NdeI and BamHI restriction endonucleases (NEB) and ligated (T4 DNA ligase, NEB) into a modified pET expression vector that had also been restricted using the aforementioned enzymes, and contained ampicillin resistance for selectivity. The vector was transformed into electrocompetent E. coli strain BL21-DE3 and transformants plated onto LB agar plates containing ampicillin (100 μg/mL) for selectivity. Transformant DNA was isolated and sent for sequencing to confirm the identity of the vector construct. Following DNA sequencing, overexpression of GabT was achieved in LB media containing ampicillin (100 μg/mL) and induction using IPTG (Isopropyl β-D-1-thiogalactopyranoside; 1 mM final concentration). The protein was purified using a HiTrap Chelating HP 5 mL column (GE Healthcare) for nickel affinity chromatography on an FPLC (Fast Protein Liquid Chromatography. GE Healthcare), exploiting an N-terminal His₆-tag on GabT. SDS-PAGE confirmed the protein size.

Protein activity was confirmed using a coupled assay that comprised GabT and a commercially available glutamate dehydrogenase (GDH; Sigma Aldrich). The assay works as illustrated below, with the dehydrogenase catalysing the oxidative deamination of glutamate—the co-product of the GabT transamination reaction. The deamination requires the use of a cofactor, NAD (nicotinamide adenine dinucleotide), which is concurrently reduced by the enzyme to NADH, and the formation of this reduced product can be detected by UV photospectrometry as a hyperchromic shift at 340 nm.

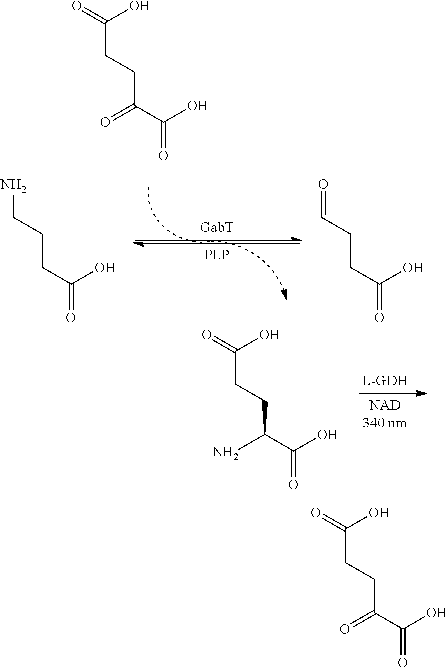

This assay was used to determine the substrate scope of GabT, which was ultimately shown to be C4-C8 omega-amino acids. Therefore, although a viable biocatalyst for these substrates had been identified, it could not produce the desired longer chain (>C9) products. With this in mind, a new strategy was adopted.

### Bioprospecting

In order to test for activity, bacterial strains were grown on minimal media containing no source of nitrogen other than supplemented ω-amino alkanoates (e.g. 8-amino octanoic acid, 11-amino undecanoic acid and 12-aminododeconic acid) covering a range a carbon chain lengths (C4-C12). The bacteria were incubated for several days at 37° C. and monitored regularly for colony formation on the surface of the agar. Without nitrogen, the bacteria would be unable to grow, and as such, any colonies on the plates would be indicative of a strain that was able to break down the ω-amino alkanoate and release the nitrogen for growth. From this screen, one bacterial strain, a Pseudomonas sp., was identified as being able to grow on the minimal media plates supplemented with C4-C12 ω-amino alkanoates, and a large culture was inoculated with the bacteria in order to test the crude cell lysate for activity and identify the type of protein acting on the C12 amino acid. Genomic DNA was also isolated from the strain and sent to the Beijing Genomics Institute for sequencing.

Assaying of the cell-free extract was suggestive, although not conclusive, that a transaminase was catalysing the breakdown of the 12-aminododecanoic acid. The screen involved the addition of both pyruvate and alpha-ketoglutarate, as well as both alanine and glutamate dehydrogenases and screening for NADH formation in the manner previously described. This assay was positive for transaminase activity.

### Identifying the Biocatalyst within Pseudomonas sp.

Following genomic sequence elucidation of the Pseudomonas sp., the genome was analysed using Exonerate—a sequence alignment tool that can align query sequences against genomic DNA and identify similar genes. In light of previous investigations with GabT, this was used as the query sequence in a protein2genome model alignment, and following analysis of the genome, fourteen potential homologues were identified in the Pseudomonas sp. genome. These ranged in sequence identity from 4-74%. Primers were designed to amplify each of the fourteen genes from the genomic DNA for cloning into E. coli.

### Cloning and Expression

Each of the gene fragments was amplified by PCR from genomic DNA, digested using NdeI and BamHI restriction endonucleases and ligated into a modified pET vector in the same manner as GabT described previously. The vector was transformed into electrocompetent E. coli BL21-DE3 and plated on LB agar plates containing ampicillin. Following incubation at 37° C. overnight, observed transformants were used to inoculate small LBAmp cultures (typically 10 mL). Vector DNA was isolated from the bacteria and sent for DNA sequencing to confirm the construct.

Protein overexpression was achieved by growing 200 mL of E. coli BL21-DE3 containing the desired vector in LB media containing ampicillin (100 μg/mL) at 37° C. When the OD₆₀₀ reached 0.6-1.0, the cultures were induced by the addition of IPTG (1 mM final concentration) and further incubated at 37° C. for 18 hours. The cells were isolated by centrifugation (4.500 rpm; 20 minutes) and the supernatant discarded. The pellet was resuspended in potassium phosphate buffer (100 mM, pH 7.5) and cell lysis was achieved using BugBuster® 10× reagent (Merck) with shaking on ice for one hour. Cellular debris was precipitated by centrifugation (18,000 rpm, 1 hour) and the cell-free extract was passed over a HiTrap Chelating HP column on an FPLC against an increasing concentration of imidazole. Eluted proteins were washed in phosphate buffer (100 mM, pH 7.5) and concentrated by centrifugation utilising spin columns (GE Healthcare; 10k MWCO). Purity was assessed by SDS-PAGE.

### Assaying For Novel Activity

Activity for each protein was assessed using coupled dehydrogenase assays as described previously. A typical assay comprised:

Substrate (6.25 mM final concentration in potassium phosphate buffer)

Co-substrate (pyruvate (0.5 mM final concentration) or α-KG (0.25 mM final concentration)

NAD (1.25 mM final concentration)

Dehydrogenase (1 μL from ADH-≧35 units/mL stock solution or GDH≧35 units/mg protein stock)

Transaminase (final concentration dependent)

Potassium phosphate buffer (100 mM)

UV absorbance at 340 nm was recorded at 28° C. over a pH range of 7.5-10 with a range of substrates. Of the fourteen proteins tested, three referred to herein as p6 (amino acid sequence provided in SEQ ID NO:1; polynucleotide sequence provided in SEQ ID NO:3), p7 (amino acid sequence provided in SEQ ID NO:2; polynucleotide sequence provided in SEQ ID NO:4), and p4 (amino acid sequence provided in SEQ ID NO:6; polynucleotide sequence provided in SEQ ID NO:14), have shown the desired activity with ω-amino acids, notably including long chain amino acids C11 and C12 (shown below left; C9 and C10 are not commercially available). In addition, the p6 enzyme also accepts substrates as small as C3 (β-alanine) and is tolerant of some functionalisation along the carbon chain. Similarly, the p7 enzyme accepts substrates as small as C4 (γ-aminobutyrate; GABA) and is tolerant of some functionalisation along the carbon chain.

The p6 enzyme is a pyruvate: ω-amino acid transaminase, with a pH optimum around pH 10. The enzyme has 19% sequence identity to GabT, and is predicted to be a Beta-alanine: pyruvate transaminase based on BLAST analysis against online protein databases. Although other proteins in the database share high homology (up to 87%, with a recent entry 07.03.13 sharing 99%, none have been characterised to date).

The p7 enzyme is a pyruvate: ω-amino acid transaminase, with a pH optimum around pH 10. The enzyme has 27% sequence identity to GabT, and is predicted to be an acetylornithine transaminase based on BLAST analysis against online protein databases.

The p4 enzyme is a pyruvate: ω-amino acid transaminase, with a pH optimum around pH 10. The enzyme has 23% sequence identity to GabT, and is predicted to be a putrescine transaminase based on BLAST analysis against online protein databases.

To corroborate the findings of the UV assay, a large-scale (200 mL) p6 reaction was carried out using C11 amino acid substrate. After seven days, the mixed reaction was allowed to settle and yellow oil was observed on the surface of the reaction. This oil was concentrated in vacuo and analysed by GC-MS. In addition to the detection of alanine (co-product for the reaction), a peak corresponding to the mass of the C11 diacid was also found in large concentrations. The diacid is thought to be the oxidation product of the semialdehyde formed by the transaminase, further supporting the findings of the UV assay.

The alanine dehydrogenase-coupled assay has been used to determine some early kinetic parameters for p6 and p7. Reactions with C3 to C8 for p6 and C4 to CS have been fully characterised and show no clear trend in terms of reaction rate vs. carbon chain length. For C11 and C12, the solubility of the substrates is extremely low, and as such, kinetic parameters cannot be determined.

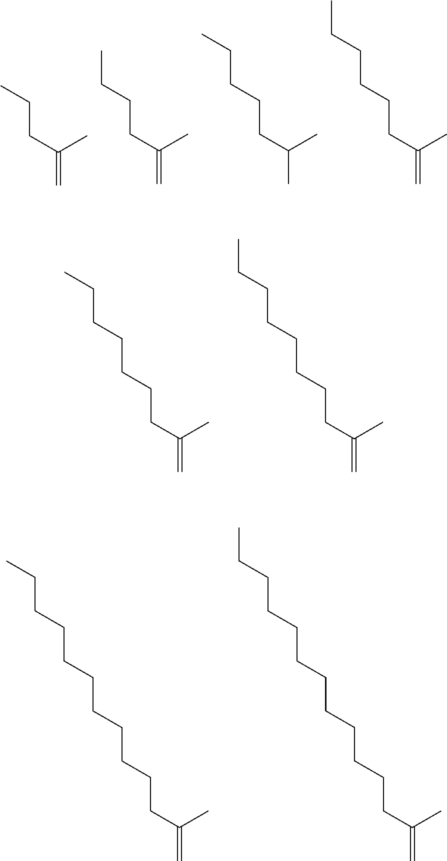

### Ancestral Reconstruction

p7N6, p7N15, p7N16, p7N17, p7N43 and p7N48 are de novo peptide sequences based on p7. These molecules were produced by ancestral reconstruction using the method described below.

Homologues of p7 were obtained by BLAST analysis of the amino acid sequence, and were aligned to one another. Multiple sequence alignment (MSA) for the sequences was inferred using MAFFT version 7.043 (Katoh and Standley, 2013) and Seaview version 4.4.1 (Gout' et al., 2010). A preliminary MSA was inferred for the data using the LINSI option of MAFFT. Using Seaview, the preliminary alignment was refined to yield the final alignment. Duplicate sequences were identified, and then removed from the final alignment using IQ-TREE version 0.9.3 (Minh et al., 2013). The reduced data was realigned.

To assess whether the data could be assumed to have evolved under globally stationary. reversible, and homogeneous (SRH) conditions (Jayaswal et al. 2011), the inventors surveyed the alignment using Homo version 1.0 (available at http://www.bioinformatics.csiro.au/homo) (Ababneh et al., 2006). The observed p-values were plotted against the expected p-values obtained under the null distribution. Forty-five of the 18336 tests (˜0.2%) produced a p-value<0.05 and the smallest p-value was 0.0126, implying that the data are consistent with evolution under globally SRH conditions.

IQ-TREE version 0.9.3 (Minh et al., 2013) was used to identify optimal models of evolution. The optimal model of evolution for the master alignment was identified using the -m TESTONLY option with IQ-TREE invoked and found to be the LG+I+G4 model. A phylogenetic tree for the master alignment was inferred using maximum-likelihood (ML), as implemented in IQ-TREE. A non-parametric bootstrap analysis was carried out using the UFBoot method (using default settings) and the inferred trees were then used to infer the ancestral sequences. Ancestral amino-acid sequences were inferred under the ML criterion using FastML (Pupko et al., 2002) and the following ancestral sequences were then identified and chosen for further biochemical analysis;

p7N6 which shares 91% sequence identity with p7 and 25% identity to GabT (amino acid sequence of p7N6provided in SEQ ID NO:7; polynucleotide sequence provided in SEQ ID NO:15),

p7N15 which shares 85% sequence identity with p7 and 27% identity to GabT (amino acid sequence of p7N15 provided in SEQ ID NO:8; polynucleotide sequence provided in SEQ ID NO:16),

p7N16 which shares 81% sequence identity with p7 and 27% identity to GabT (amino acid sequence of p7N16provided in SEQ ID NO:9; polynucleotide sequence provided in SEQ ID NO:17),

p7N17 which shares 80% sequence identity with p7 and 27% identity to GabT (amino acid sequence of p7N17 provided in SEQ ID NO:10 polynucleotide sequence provided in SEQ ID NO:18),

p7N43 which shares 77% sequence identity with p7 and 27% identity to GabT (amino acid sequence of p7N43 provided in SEQ ID NO:11; polynucleotide sequence provided in SEQ ID NO:19), and

p7N48 which shares 76% sequence identity with p7 and 27% identity to GabT (amino acid sequence of p7N43 provided in SEQ ID NO:12; polynucleotide sequence provided in SEQ ID NO:20).

Proteins encoded by p7N6, p7N15, p7N16, p7N17, p7N43 and p7N48 were expressed using an E. coli recombinant expression platform and subsequently purified. Proteins were tested using the same coupled assays described above and found to have activity in accordance with p7.

### Functional Characterisation

Each of the proteins were assayed for substrate specificity. Substrates identified by assaying for each of the proteins are summarized below.

p4—6-aminohexanoic acid, 7-aminoheptanoic acid, 8-aminooctanoic acid, 11-aminoundecanoic acid, 12-aminododecanoic acid, putrescine, cadaverine, 3-aminocyclohexanoate, propionaldehyde, butyraldehyde, tyramine, 2-aminoindan, 2-methylbenzylamine, hexamethylenediamine, 1,7-diaminoheptane, 1,8-diaminooctane, 1,9-diaminononane, 1,10-diaminodecane, cyclohexylamine, 6-aminohexanol, 7-aminoheptanol, 8-aminooctanol, 9-aminononanol, 10-aminodecanol, cyclohexanone, dopamine, serotonin, alanine and pyruvate,

p6—glycine, beta-alanine, 4-aminobutyrate, 5-aminopentanoate, 6-aminohexanoic acid, 7-aminoheptanoic acid, 8-aminooctanoic acid, 11-aminoundecanoic acid, 12-aminododecanoic acid, 3-aminoisobutyrate, putrescine, cadaverine, 3-aminocyclohexanoate, propionaldehyde, butyraldehyde, tyramine, 2-aminoindan, hexamethylenediamine, 1,7-diaminoheptane, 1,8-diaminooctane, 1,9-diaminononane, 1,10-diaminodecane, 6-aminohexanol, 7-aminoheptanol, 8-aminooctanol, 9-aminononanol, 10-aminodecanol , taurine, glyceraldehyde, 3-aminoheptanoic acid, cyclohexylamine, 2-methylbenzylamine, dihydroxyacetone phosphate, hydroxymethylfurfural, ethanolamine, alanine and pyruvate.

p7—glycine, 4-aminobutyrate, 5-aminopentanoate, 6-aminohexanoic acid, 7-aminoheptanoic acid, 8-aminooctanoic acid, 11-aminoundecanoic acid, 12-aminododecanoic acid, 4-amino-2-hydroxybutyrate, putrescine, cadaverine, hexamethylenediamine, 1,7-diaminoheptane, 1,8-diaminooctane, 1,9-diaminononane, 1,10-diaminodecane, 6-aminohexanol, 7-aminoheptanol, 8-aminooctanol, 9-aminononanol, 10-aminodecanol, 2,4-diaminobutyrate, 2-methylbenzylamine, dihydroxyacetone phosphate, hydroxymethylfurfural, alanine and pyruvate.

p7N6—glycine, 4-aminobutyrate, 5-aminopentanoate, 6-aminohexanoic acid, 7-aminoheptanoic acid, 8-aminooctanoic acid, 11-aminoundecanoic acid, 12-aminododecanoic acid, ornithine, lysine, 3-aminocyclohexanoate, 4-amino-2-hydroxybutyrate, putrescine, cadaverine, hexamethylenediamine, 1,7-diaminoheptane, 1,8-diaminooctane, 1,9-diaminononane, 1,10-diaminodecane, 6-aminohexanol, 7-aminoheptanol. 8-aminooctanol, 9-aminononanol, 10-aminodecanol, 2,4-diaminobutyrate, 2-methylbenzylamine, dihydroxyacetone phosphate, hydroxymethylfurfural, alanine and pyruvate.

p7N15—glycine, 4-aminobutyrate, 5-aminopentanoate, 6-aminohexanoic acid, 7-aminoheptanoic acid, 8-aminooctanoic acid, 11-aminoundecanoic acid, 12-aminododecanoic acid, ornithine, lysine, 3-aminocyclohexanoate, 4-amino-2-hydroxybutyrate, putreseine, cadaverine, diethylaminomalonate, hexamethylenediamine, 1,7-diaminoheptane, 1,8-diaminooctane, 1,9-diaminononane, 1,10-diaminodecane, 6-aminohexanol, 7-aminoheptanol, 8-aminooctanol, 9-aminononanol, 10-aminodecanol, 2,4-diaminobutyrate, 2-methylbenzylamine, dihydroxyacetone phosphate, hydroxymethylfurfural, alanine and pyruvate.

p7N16—glycine, 4-aminobutyrate, 5-aminopentanoate, 6-aminohexanoic acid, 7-aminoheptanoic acid, 8-aminooctanoic acid, 11-aminoundecanoic acid, 12-aminododecanoic acid, ornithine, lysine, 3-aminocyclohexanoate, 4-amino-2-hydroxybutyrate, putrescine, cadaverine, N-acetyl-L-ornithine, hexamethylenediamine, 1,7-diaminoheptane, 1,8-diaminooctane, 1,9-diaminononane, 1,10-diaminodecane, 6-aminohexanol, 7-aminoheptanol, 8-aminooctanol, 9-aminononanol, 10-aminodecanol, 2,4-diaminobutyrate, 2- methylbenzylamine, dihydroxyacetone phosphate, hydroxymethylfurfural, alanine and pyruvate.

p7N17—glycine, 4-aminobutyrate, 5-aminopentanoate, 6-aminohexanoic acid, 7-aminoheptanoic acid, 8-aminooctanoic acid, 11-aminoundecanoic acid, 12-aminododecanoic acid, ornithine, lysine, 3-aminocyclohexanoate, 4-amino-2-hydroxybutyrate, putrescine, cadaverine, N-acetyl-L-ornithine, hexamethylenediamine, 1,7-diaminoheptane, 1,8-diaminooctane, 1,9-diaminononane, 1,10-diaminodecane, 6-aminohexanol, 7-aminoheptanol, 8-aminooctanol, 9-aminononanol, 10-aminodecanol, 2,4-diaminobutyrate, 2- methylbenzylamine, dihydroxyacetone phosphate, hydroxymethylfurfural, alanine and pyruvate.

p7N43—glycine, 4-aminobutyrate, 5-aminopentanoate, 6-aminohexanoic acid, 7-aminoheptanoic acid, 8-aminooctanoic acid, 11-aminoundecanoic acid, 12-aminododecanoic acid, ornithine, lysine, 3-aminocyclohexanoate, 4-amino-2-hydroxybutyrate, putrescine, cadaverine, N-acetyl-L-ornithine, diethylaminomalonate, cyclohexylamine, hexamethylenediamine, 1,7-diaminoheptane, 1,8-diaminooctane, 1,9-diaminononane, 1,10-diaminodecane, 6-aminohexanol, 7-aminoheptanol, 8-aminooctanol, 9-aminononanol, 10-aminodecanol, 2,4-diaminobutyrate, 2-methylbenzylamine, dihydroxyacetone phosphate, hydroxymethylfurfural, alanine and pyruvate.

p7N48—glycine, 4-aminobutyrate, 5-aminopentanoate, 6-aminohexanoic acid, 7-aminoheptanoic acid, 8-aminooctanoic acid, 11-aminoundecanoic acid, 12-aminododecanoic acid, ornithine, lysine, 3-aminocyclohexanoate, 4-amino-2-hydroxybutyrate, putrescine, cadaverine, N-acetyl-L-ornithine, diethylaminomalonate, cyclohexylamine, hexamethylenediamine, 1,7-diaminoheptane, 1,8-diaminooctane, 1,9-diaminononane, 1,10-diaminodecane, 6-aminohexanol, 7-aminoheptanol, 8-aminooctanol, 9-aminononanol, 10-aminodecanol, 2,4-diaminobutyrate, 2-methylbenzylamine, dihydroxyacetone phosphate, hydroxymethylfurfural, alanine and pyruvate.

### X-Ray Crystallography

The p6 protein was crystallised from purified P6 at 8 mg/mL in 50 mM potassium phosphate buffer using a reservoir solution of 200 mM MgCl, 100 mM Tris buffer at pH 7 and 10% PEG 8000 at 20 C. Crystals were taken to the Australian Synchrotron and the subsequent data were indexed using XDS (Kabsch, 2010). The data were scaled using the CCP4 program suite and the structure was solved using molecular replacement using PDB code 3A8U as a starting model and the program Phaser (McCoy et al., 2007). The structure was subsequently rebuilt and refined using Coot (Emsley et al., 2010) and Refmac (Mushudov et al., 2011) (Appendix I).

The structure of p7 was determined from a crystal grown using 5 mg/mL p7 protein (in PO4/NaCl) from a solution containing 1.61 M ammonium sulfate, 0.05 M sodium MES pH 6.8, 0.9% v/v dioxane. The structure of P7n6 was determined from a crystal grown using 10 mg/mL protein (in sodium ADA/NaCl) from a solution containing 0.155 M magnesium chloride, 0.1 M tris chloride pH 8.5, 12.8% v/v glycerol and 15.2% w/v PEG 8000. The structure of P7n15 was determined from a crystal grown using 10 mg/mL protein (in sodium ADA/NaCl) from a solution containing 0.152 M magnesium chloride, 0.1 M tris chloride pH 7.2. 17.1% v/v glycerol and 14.9% w/v PEG 8000. The structure of P7n16 was determined from a crystal grown using 10 mg/mL protein (in tris chloride/NaCl) from a solution containing 0.135 M ammonium formate, and 20.1% w/v PEG 3350. The structure of P7n17 was determined from a crystal grown using 10 mg/mL protein (in tris chloride/NaCl) from a solution containing 0.152 M magnesium chloride, 0.1 M tris chloride pH 7.1. 14.8% v/v glycerol and 11.9% w/v PEG 8000. The structure of P7n43 was determined from a crystal grown using 4 mg/mL protein (in sodium MOPS/NaCl) from a solution containing 0.011 M calcium acetate, 0.1 M tris chloride pH 7.1 and 15.1% w/v PEG 8000. The structure of P7n48 was determined from a crystal grown using 10 mg/mL protein (in PO4/NaCl) from a solution containing 0.03 M magnesium nitrate, and 15.6% w/v PEG 3350. The p7, p7N6, p7N15, p7N16, p7N16, p7N17, p7N43 and p7N48 structures have been solved (data not shown) and are similar to p6. The RMS values for each, relative to p6 are shown below.

p6,p7—1.106

p6, p7N6—1.094

p6,p7N 15—1.076

p6,p7N16—1.076

p6,p7N 17—1.054

p6,p7N43—1.076

p6,p7N48—1.114

Crystals were taken to the Australian Synchrotron and the subsequent data were indexed using XDS (Kabsch, 2010). The data were scaled using the CCP4 program suite and the native structure was solved using molecular replacement using PDB code 3GJU as a starting model and the program Phaser (McCoy et al., 2007). The structure was subsequently rebuilt and refined using Coot (Emsley et al., 2010) and Refmac (Mushudov et al., 2011). The various ancestral protein structures (nodes n6 to n48) were solved using the same procedure but with the native structure as the starting point for molecular replacement.

To corroborate the findings of the UV assay, a large-scale (200 mL) p6 reaction was carried out using C11 amino acid substrate. After seven days, the mixed reaction was allowed to settle and yellow oil was observed on the surface of the reaction. This oil was concentrated in vacuo and analysed by GC-MS. In addition to the detection of alanine (co-product for the reaction), a peak corresponding to the mass of the C11 diacid was also found in large concentrations. The diacid is thought to be the oxidation product of the semialdehyde formed by the transaminase, further supporting the findings of the UV assay.

The alanine dehydrogenase-coupled assay has been used to determine some early kinetic parameters for all the aforementioned enzymes. Reactions with C3 to C8 for p6 and C4 to C8 for p7 have been fully characterised and show no clear trend in terms of reaction rate vs. carbon chain length. For C11 and C12, the solubility of the substrates is extremely low, and as such, kinetic parameters cannot be determined.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive. The present application claims priority from AU 2013902128 filed 12 Jun. 2013, the entire contents of which are incorporated herein by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Ababneh et al. (2006) Bioinformatics 22: 1225-1231,

Bernt and Bergmeyer (1974) Methoden Enzym. 3(2):1749-53,

Cadwell and Joyce (1992) PCR Methods Appl. 2:28-33,

Capecchi (1980) Cell 22:479-488,

Clapp (1993) Clin. Perinatol. 20:155-168,

Coco et al. (2001) Nature Biotechnology 19:354-359,

Coco et al. (2002) Nature Biotechnology 20:1246-1250,

Crameri et al. (1998) Nature 391:288-291,

Curiel et al. (1992) Hum. Gen. Ther. 3:147-154,

Dunbrack et al. (1997) Folding and Design 2:R27-R42,

Eggert et al. (2005) Chemobiochem. 6:1062-1067,

Eglitis et al. (1988) Biotechniques 6:608-614,

Emsley et al. (2010) Acta Crystallogr. D Biol. Crystallogr. 66:486-501,

Gordon et al. (1999) Chemical-Biological Interactions 14:463-470,

Gouy et al. (2010) Molecular Biology and Evolution 27:221-224,

Graham et al. (1973) Virology 54:536-539,

Harayama (1998) Trends Biotechnol. 16:76-82,

Haseloff and Gerlach (1988) Nature 334:585-591,

Hellinga (1997) Proc Natl Acad Sci USA 94:10015-10017,

Hinchee et al. (1988) Biotechnology 6:915-922,

Jayaswal et al. (2011) Systematic Biology 60:74-86,

Kabsch (2010) Acta Crystallogr. Sect. D-Biol. Crystallogr. 66:125-132,

Katoh K and Standley (2013) Molecular Biology and Evolution 30:772-780,

Koziel et al. (1996) Plant Mol. Biol. 32:393-405,

LeJeune et al. (1998). Nature 395:27-28,

Leung et al. (1989) Technique 1:11-15,

Lu et al. (1993) J. Exp. Med. 178:2089-2096,

Malik et al. (2012) Appl. Microbiol. Biotechnol. 94:1163-1171,

McCoy (2007) J. Appl. Crystallogr. 40 :658-674,

Mehta et al. (1993) Eur. J. Biochem. 214:549-561,

Minh et al. (2013) Molecular Biology and Evolution 30:1188-1195,

Murshudov et al. (2011) Acta Crystallogr. D Biol. Crystallogr. 67:355-367,

Needleman and Wunsch (1970) J. Mol. Biol. 48:443-453,

Ness et al. (2002) Nature Biotechnology 20:1251-1255,

Niedz et al. (1995) Plant Cell Reports 14:403-406,

Ostermeier et al. (1999) Nat Biotechnol. 17:1205-1209,

Ow et al. (1986) Science 234:856-859,

Petrikovics et al. (2000a) Toxicology Science 57: 16-21,

Petrikovics et al. (2000b) Drug Delivery 7: 83-89,

Pupko et al. (2002) Bioinformatics 18:1116-1123,

Prasher et al. (1985) Biochem. Biophys. Res. Commun. 127:31-36,

Sieber et al. (2001) Nature Biotechnology 19:456-460,

Stalker et al. (1988) Science 242: 419-423,

Stemmer (1994a) Proc. Natl. Acad. Sci. USA 91:10747-10751.

Stemmer (1994b) Nature 370 (6488):389-391.

Thillet et al. (1988) Journal of Biological Chemistry 263:12500-12508,

Volkov et al. (1999) Nucleic Acids Research 27:e18i-vi,

Wagner et al. (1992) Proc. Natl. Acad. Sci. USA 89:6099-6103,

Zhao et al. (1998) Nature Biotechnology 16:258261.

Lengthy table referenced here
US20160208226A1-20160721-T00001
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20160208226A1). An electronic copy of the table will also be available from the USPTO upon request and payment of the fee set forth in 37 CFR 1.19(b)(3).

1. A substantially purified and/or recombinant polypeptide comprising:
i) an amino acid sequence as provided in any one of SEQ ID NOs:1, 2 or 6 to 12,
ii) an amino acid sequence which is at least 40% identical to any one or more of SEQ ID NOs:1, 2 or 6 to 12, or
iii) a biologically active fragment of i) or ii),
wherein the polypeptide has amino transferase activity.

2. The polypeptide of claim 1, wherein the polypeptide catalyses the transfer of an amino group from an amino donor to an amino acceptor.

3. The polypeptide of claim 2, wherein the polypeptide catalyses the reversible transfer of an amino group from an amino donor to an amino acceptor.

4. The polypeptide of claim 1, wherein the amino donor or amino acceptor comprises 3 to 12 carbons.

5. The polypeptide of claim 1, wherein the amino donor or amino acceptor comprises 4 to 12 carbons.

6. The polypeptide of claim 1, wherein the amino donor or amino acceptor comprises 9 to 12 carbons.

7. The polypeptide of claim 2, wherein the amino donor is an amino acid or an amine compound.

8. The polypeptide of claim 7, wherein the amino acid is an α-amino acid or an ω-amino acid and/or the amine compound is a diamine.

9.-11. (canceled)

12. The polypeptide of claim 1, wherein the amino acceptor is a keto acid, a ketone or an aldehyde.

13. (canceled)

14. An isolated and/or exogenous polynucleotide comprising one or more of:
i) a sequence of nucleotides as provided in any one or more of SEQ ID NOs:3 to 5, or 14 to 20,
ii) a sequence of nucleotides encoding a polypeptide of claim 1,
iii) a sequence of nucleotides which is at least 45% identical to i),
iv) a sequence of nucleotides which hybridizes to i) under stringent conditions, or
v) a sequence of nucleotides complementary to any one of i) to iv).

15. The polynucleotide of claim 14 which encodes a polypeptide that has amino transferase activity.

16. A vector comprising an exogenous polynucleotide of claim 14 encoding a polypeptide comprising:
i) an amino acid sequence as provided in any one of SEQ ID NOs:1, 2 or 6 to 12,
ii) an amino acid sequence which is at least 40% identical to any one or more of SEQ ID NOs:1, 2 or 6 to 12, or
iii) a biologically active fragment of i) or ii),
wherein the polypeptide has amino transferase activity, and wherein the polynucleotide is operably linked to a heterologous promoter.

17. A host cell comprising an exogenous polynucleotide of claim 14 encoding a polypeptide comprising:
i) an amino acid sequence as provided in any one of SEQ ID NOs:1, 2 or 6 to 12,
ii) an amino acid sequence which is at least 40% identical to any one or more of SEQ ID NOs:1, 2 or 6 to 12, or
iii) a biologically active fragment of i) or ii),
wherein the polypeptide has amino transferase activity, and wherein the polynucleotide is operably linked to a heterologous promoter.

18. A method of producing a polypeptide, the method comprising cultivating a host cell of claim 17 encoding said polypeptide under conditions which allow expression of the polynucleotide encoding the polypeptide, and recovering the expressed polypeptide.

19. (canceled)

20. (canceled)

21. A transgenic non-human organism comprising an exogenous polynucleotide encoding a polypeptide of claim 1.

22. An extract of a host cell of claim 17, wherein the extract comprises a polypeptide comprising:
i) an amino acid sequence as provided in any one of SEQ ID NOs:1, 2 or 6 to 12,
ii) an amino acid sequence which is at least 40% identical to any one or more of SEQ ID NOS:1, 2 or 6 to 12, or
iii) a biologically active fragment of i) or ii),
wherein the polypeptide has amino transferase activity.

23. (canceled)

24. A composition for catalysing transfer of an amino group from an amino donor to an amino acceptor, the composition comprising a substantially purified and/or recombinant polypeptide:
of claim 1.

25. A method for catalysing transfer of an amino group from an amino donor to an amino acceptor, the method comprising contacting the amino donor and amino acceptor with a polypeptide of claim 1.

26. (canceled)

27. A method for catalysing transfer of an amino group from an amino donor to an amino acceptor with a substantially purified and/or recombinant polypeptide which has amino transferase activity, wherein the amino donor or amino acceptor comprises at least 9 carbons.

28.-31. (canceled)

32. A method of producing a polypeptide with enhanced ability to catalyse the transfer of an amino group from an amino donor to an amino acceptor, or having altered substrate specificity, the method comprising:
i) altering one or more amino acids of a polypeptide of claim 1,
ii) determining the ability of the altered polypeptide obtained from step i) to catalyse the transfer of an amino group from the amino donor to the amino acceptor, and
iii) selecting an altered polypeptide with enhanced ability to catalyse the transfer of an amino group from the amino donor, or having altered substrate specificity, when compared to the polypeptide used in step i).

33. (canceled)

34. A method for screening for a microorganism capable of catalysing the transfer of an amino group from an amino donor which comprises at least 9 carbons, the method comprising:
i) culturing a candidate microorganism in the presence of an amino donor which comprises at least 9 carbons, as a sole nitrogen source, and
ii) determining whether the microorganism is capable of growth and/or division.

35.-43. (canceled)

44. A composition for catalysing transfer of an amino group from an amino donor to an amino acceptor, the composition comprising a host cell of claim 17.


SEQUENCE LISTING (20 sequences)

SEQ ID NO: 1 - protein, 468 aa, Pseudomonas sp.
MGSSHHHHHHSSGLVPRGSHMNQQVNVAPSAAADLNLKAHWMPFSANRNFHKDPRIIVAAEGSWLVDDKGRRIYDSLSGLWTCGAGHSRKEIADAVAKQIGTLDYSPGFQYGHPLSFQLAEKIAQMTPGTLDHVFFTGSGSECADTSIKMARAYWRIKGQAQKTKLIGRARGYHGVNVAGTSLGGIGGNRKMFGPLMDVDHLPHTLQPGMAFTKGAAETGGVELANELLKLIELHDASNIAAVIVEPMSGSAGVIVPPKGYLQRLREICDANDILLIFDEVITAFGRMGKATGAEYFGVTPDIMNVAKQVTNGAVPMGAVIASSEIYDTFMNQNLPEYAVEFGHGYTYSAHPVACAAGIAALDLLQKENLIQQSAELAPHFEKALHGLKGTKNVIDIRNCGLAGAIQIAARDGDAIVRPFEASMKLWKEGFYVRFGGDTLQFGPTFNAKPEDLDRLFDAVGEALNGVA

SEQ ID NO: 2 - protein, 479 aa, Pseudomonas sp.
MGSSHHHHHHSSGLVPRGSHMTDYAKLFEQDRAHFMHPSTHAHDHASGALPGRIITGASGVRIRDHQGRELLDAFAGLYCVNIGYGRLEVADAIHEQAKQLAYYHTYVGHASEAIIELSARIIRDWAPAGMKKVYYGLSGSDANETQVKLVRYYNNVLGRPQKKKIISRQRGYHGSGIVTGSLTGLASFHQHFDLPVEGVKHTLCPHFYKAPAGMDEAAFVRHCAQELENLILAEGPDTVAAFIGEPVMGTGGIIVPPKGYWEAIQAVLAKYDVLLIADEVVCAFGRLGDKMGSQRHAMRPDLITTAKGLTSAYAPLSAVIVGEKVWDVIDSASTREGAMGHGWTYSGHPICAAAALANLDILERENITANAADVGGYLNQQLRQAFEGHPLVGEVRGDGMLAALEFMADREARTPFDAALKVGPKVSAACLERGMIARAMPHGDILGFAPPLVLSRAEADEVVGIAKAAVDAVAAEVL

SEQ ID NO: 3 - DNA, 1347 bp, Pseudomonas sp.
atgaaccagcaagtgaacgtagcgccgtcggccgccgccgacctgaacctgaaggcccactggatgcccttcagcgccaaccgcaacttccacaaggacccgcgcatcatcgtggccgccgagggcagctggctggtggacgacaagggccggcgcatctacgacagcctgtccggcctgtggacctgcggcgccggtcactcgcgcaaggaaatcgccgacgcggtggccaagcagattggcaccctcgactactccccgggcttccagtacggccacccgctgtccttccagctggccgagaagatcgcccagatgacccccggcaccctcgaccacgtgttcttcaccggctccggttccgagtgcgccgacacctcgatcaagatggcccgcgcctactggcgcatcaaaggccaggcgcagaagaccaagctgatcggccgcgcccgtggctaccacggcgtgaacgtcgccggcacctccctgggcggcatcggcggcaaccgcaagatgttcggcccgctgatggacgtcgaccacctgccgcacaccctgcagccgggcatggcctttaccaagggtgcggccgagaccggcggcgtcgagctggccaacgaactgctgaagctgatcgagctgcacgacgcctccaacatcgccgcggtgatcgtcgagccgatgtccggctccgccggcgtgatcgtgccgccgaagggctacctgcagcgcctgcgggaaatctgcgacgccaacgacatcctgctgatcttcgacgaagtcatcaccgccttcggccgcatgggcaaggccaccggcgccgaatacttcggcgtgaccccggacatcatgaacgtcgccaagcaggtcaccaacggcgccgtgcccatgggcgcggtgatcgccagcagcgaaatctacgacaccttcatgaaccagaacctgccggaatacgcggtggagttcggccatggctacacctactccgcgcacccggtcgcctgcgccgccggcatcgccgcgctggacctgctgcagaaggaaaacctgatccagcagtccgccgaactggcgccgcacttcgagaaggccctgcacggcctcaagggcacgaagaacgtcatcgacatccgcaactgcggcctggccggcgccatccagatcgccgcccgcgacggcgacgccatcgtccgcccgttcgaagccagcatgaagctgtggaaggaaggcttctacgtgcgcttcggcggcgacaccctgcagttcgggccgaccttcaacgccaagcccgaagacctcgaccgcctgttcgacgcggtcggcgaagccctcaacggggtggcgtaa

SEQ ID NO: 4 - DNA, 1380 bp, Pseudomonas sp.
atgaccgactacgccaagctgttcgagcaggaccgtgcgcacttcatgcacccctccacccacgcccacgaccacgccagcggcgccctgcccgggcgcatcatcaccggcgcctcgggcgtgcgcatccgcgaccaccagggccgcgagctgctggatgccttcgccgggctgtactgcgtgaacatcggctacggccgccttgaggtggccgacgccatccatgagcaggccaagcagctggcctactaccacacctacgtcggccacgcgtccgaggcgatcatcgagctgtccgcgcggatcatccgcgactgggcgccggcgggcatgaagaaggtctactacggcctctccggctccgacgccaacgaaacccaggtcaagctggtgcgctactacaacaacgtgctggggcggccgcagaagaagaagatcatctcgcgccagcgcggctaccacggctcgggcatcgtcaccggcagcctcaccggactggccagcttccaccagcacttcgacctgccggtggaaggcgtcaaacacaccctctgcccgcacttctacaaggccccggcgggcatggacgaggcggccttcgtgcgccactgcgcccaggagctggagaacctgatcctcgccgagggcccggacaccgtggcggccttcatcggcgagccggtgatgggcaccggcggcatcatcgtcccgcccaagggctactgggaggcgatccaggcggtgctggcgaagtacgacgtgctgctgatcgccgacgaggtggtctgcgccttcggccgcctcggcgacaagatgggcagccagcgccatgccatgcgcccggacctgatcaccaccgccaagggcctgaccagcgcctacgcgccgctgtccgcggtgatcgtcggcgagaaggtctgggatgtgatcgacagcgcctccacccgcgaaggcgccatgggccacggctggacctattccggccacccgatctgcgcggcggcggcgctggccaacctggacatcctcgagcgcgagaacatcaccgccaacgcggcggacgtcggcggctacctcaaccagcagttgcgccaggccttcgaaggccacccgctggtgggcgaggtgcgcggcgacggcatgctggcggcgctggagttcatggccgaccgcgaggcgcgcacgcctttcgacgcggcgctgaaggtgggtccgaaggtctcggcggcgtgcctggagcgcggcatgatcgcccgcgccatgccccacggcgacatcctcggcttcgccccgccgctggtactcagccgcgccgaggcggacgaggtggtgggcatcgccaaggccgcggtggacgcggtggcggcggaggtgctttga

SEQ ID NO: 5 - DNA, 1380 bp, Artificial Sequence
  Codon optimized coding sequence
atgaccgactacgccaagctgttcgagcaggaccgtgcgcacttcatgcacccctccacccacgcccacgaccacgccagcggcgccctgcccgggcgcatcatcaccggcgcctcgggcgtgcgcatccgcgaccaccagggccgcgagctgctggatgccttcgccgggctgtactgcgtgaacatcggctacggccgccttgaggtggccgacgccatccatgagcaggccaagcagctggcctactaccacacctacgtcggccacgcgtccgaggcgatcatcgagctgtccgcgcggatcatccgcgactgggcgccggcgggcatgaagaaggtctactacggcctctccggctccgacgccaacgaaacccaggtcaagctggtgcgctactacaacaacgtgctggggcggccgcagaagaagaagatcatctcgcgccagcgcggctaccacggctcgggcatcgtcaccggcagcctcaccggactggccagcttccaccagcacttcgacctgccggtggaaggcgtcaaacacaccctctgcccgcacttctacaaggccccggcgggcatggacgaggcggccttcgtgcgccactgcgcccaggagctggagaacctgatcctcgccgagggcccggacaccgtggcggccttcatcggcgagccggtgatgggcaccggcggcatcatcgtcccgcccaagggctactgggaggcgatccaggcggtgctggcgaagtacgacgtgctgctgatcgccgacgaggtggtctgcgccttcggccgcctcggcgacaagatgggcagccagcgccatgccatgcgcccggacctgatcaccaccgccaagggcctgaccagcgcctacgcgccgctgtccgcggtgatcgtcggcgagaaggtctgggatgtgatcgacagcgcctccacccgcgaaggcgccatgggccacggctggacctattccggccacccgatctgcgcggcggcggcgctggccaacctggacatcctcgagcgcgagaacatcaccgccaacgcggcggacgtcggcggctacctcaaccagcagttgcgccaggccttcgaaggccacccgctggtgggcgaggtgcgcggcgacggcatgctggcggcgctggagttcatggccgaccgcgaggcgcgcacgcctttcgacgcggcgctgaaggtgggtccgaaggtctcggcggcgtgcctggagcgcggcatgatcgcccgcgccatgccccacggcgacatcctcggcttcgccccgccgctggtactcagccgcgccgaggcggacgaggtggtgggcatcgccaaggccgcggtggacgcggtggcggcggaggtgctttaa

SEQ ID NO: 6 - protein, 454 aa, Pseudomonas sp.
MTKQTSAQTQHWQALSREHHLAPFTDYKQLNEKGARIITKAEGVYLWDSEGNKILDGMAGLWCMNVGYGRKELAEVAYKQMLELPYYNLFFQTAHPPALELAKAIADIAPEGMNHVFFTGSGSESNDTVLRMVRHYWSIKGKPQKKVVIGRWNGYHGSTVAGVSLGGMKALHSQGDLPIPGIVHIAQPYWYGEGGDMSAEEFGVWAAEQLEKKILEVGEENVAAFIAEPIQGAGGVIVPPDTYWPKIREILAKYEILFIADEVICGFGRTGEWFGSQYYGNAPDLMPIAKGLTSGYIPMGGVIVRDEIVDTLNEGGEFYHGFTYSGHPVAAAVALENIRILREEKIVETVKAETAPYLQKRWQELADHPLVGEARGVGMVGALELVKNKKTRERFENGVGMLCREHCFRNGLIMRAVGDTMIISPPLVITKPEIDELITLARKCLDQTAAVALS

SEQ ID NO: 7 - protein, 459 aa, Artificial Sequence
  Molecule produced by ancestral reconstruction
MTDFDQLFEQDRAHFMHPSTHAHDHASGALPGRIITGASGIRIRDHEGRELIDAFAGLYCVNIGYGRTEVADAIYKQAKELAYYHTYVGHSTEAIIELSSRIIRDWAPAGMKKVYYGLSGSDANETQIKLVRYYNNVLGRPQKKKIISRQRGYHGSGIMTGSLTGLPSFHQHFDLPVEGIKHTVCPHWYKAPAGMDEAAFVRYCADELEKLILAEGPDTVAAFIGEPVMGTGGIIVPPKGYWEAIQAVLNKYDVLLIADEVVCAFGRLGSKMGSQRYGMRPDLITTAKGLTSAYAPLSAVIVGEKVWDVIEKASQKEGAMGHGWTYSGHPICAAAALANLDILERENLTANAADVGAYLNQRLRETFEGHPLVGEVRGDGMLAALEFMADREARTPFDPALKVGPKVSAACLEDGMIARAMPHGDILGFAPPLVLTRAEADEIVGIAKAAVDEVAGEVL

SEQ ID NO: 8 - protein, 459 aa, Artificial Sequence
  Molecule produced by ancestral reconstruction
MTDLDQLFEMDRAHFMHPSTHAHDHASGALPGRIITGGKGIRIQDHEGREYIDAFAGLYCVNIGYGRTEVADAIYEQAKELAYYHTYVGHSTEAIIELSSRIIRDWAPAGMKKVYYGMSGSDANETQIKLVWYYNNVLGRPQKKKIISRQRGYHGSGIMTGSLTGLPSFHQHFDLPIERIKHTVCPHWYKAPAGMSEAQFVRYCADELEKLILAEGPDTVAAFIGEPVMGTGGIIPPPQGYWEAIQAVLNKYDILLIADEVVCGFGRLGSKMGSQHYGMKPDLITVAKGLTSAYAPLSGVIVGEKVWDVIEKGSQEHGPMGHGWTYSGHPICAAAALANLDILERENLTGNAADVGAYLQQRLRETFGGHPLVGEVRGVGMLAALEFMADKDARTPFDPALKVGPKVSAACLEDGMIARAMPHGDILGFAPPLVITRAEVDEIVGIAKQAVDEVADEVL

SEQ ID NO: 9 - protein, 459 aa, Artificial Sequence
  Molecule produced by ancestral reconstruction
MQSLDQLFEMDRAHFMHPSTHAHDHASGALPGRIITGGKGIRIEDHEGREYIDAFAGLYCVNIGYGRTEVADAIYEQAKQLAYYHTYVGHSTDAIIELSSRIIRDWAPAGMKKVYYGMSGSDANETQIKLVWYYNNVLGRPNKKKIISRERGYHGSGIVTGSLTGLPSFHQHFDLPIDRVKHTVCPHWYRAPAGMSEAQFVAYCVEELEKLIAREGADTIAAFIAEPVMGTGGIIPPPQGYWEAIQAVLRKHDILLIADEVVCGFGRLGSKMGSQHYGIKPDLITVAKGLTSAYAPLSGVIVGEKVWDVIEKGSQEHGPMGHGWTYSGHPICAAAALANLDILERENLTGNAADVGAYLQQRLHEAFGAHPLVGEVRGVGMLAALEFMADKDARTPFDPALKVGPKVSAAALEDGMIARAMPHGDILGFAPPLVTTRAEVDEIVGIAKQAVDEVADEVL

SEQ ID NO: 10 - protein, 459 aa, Artificial Sequence
  Molecule produced by ancestral reconstruction
MTSLDQLFEEDRAHFMHPSTHAHDHASGALPGKIVTGGKGIRIEDHQGREYIDAFAGLYCVNIGYGRTEVADAIYEQAKQLAYYHTYVGHSTDAIIELSSRIIRDWAPAGMKKVYYGMSGSDANETQIKIVWYYNNVLGRPNKKKIISRERGYHGSGIVTGSLTGLPSFHQHFDLPIDRVKHTVCPHWYKAPAGMSEAQFVAYCVEELEKLIAREGADTIAAFIAEPVMGTGGIIAPPQGYWEAIQAVLRKHDILLISDEVVCGFGRLGSKMGAQHYGIKPDLITVAKGLTSAYAPLSGVIVGEKVWDVIEKGSQEHGPMGHGWTYSGHPICAAAALANLDILERENLTGNAADVGAYLQQRLHEAFGAHPLVGEVRGVGMLAALEFMADKDARTPFDPALKVGPKVSAAALEDGVIARAMPHGDILGFAPPLVTTRAEVDEIVGIVKQAVDEVADEVL

SEQ ID NO: 11 - protein, 459 aa, Artificial Sequence
  Molecule produced by ancestral reconstruction
MTSLEQLLEMDRAHFMHPSTHAYDHASGALPGRIITGGKGIRIEDHEGREYIDAFAGLYCVNIGYGREEVADAIYEQAKQLAYYHTYVGHSNDPVIELSSRIIEDWAPAGMKKVFYGMSGSDANETQIKLVWYYNNVLGRPNKKKIIARERSYHGSGIVTGSLTGLPSFHQHFDLPIDRVKHTVCPHWYNAPPGMSEAQFVAYCVEELEKLIAREGADTIAAFIAEPVMGTGGIVPPPQGYWEAIQAVLRKHDILLIADEVVCGFGRLGSKTGSEHYGIKPDLITVAKGLTSAYAPLSAVIVSEKVWDVIEKGSREHGVMGHGWTYSGHPVCAAAALANLDILERENLTGNAADVGAYLQQRLHEAFGAHPLVGEVRGVGMLAALEFMADKGARTPFDPALKVSQKVAAAALEDGLIVRALPHGDILGFAPPLVTTRAEVDEIVAIAKEAFDEVADAVL

SEQ ID NO: 12 - protein, 459 aa, Artificial Sequence
  Molecule produced by ancestral reconstruction
MTDTDDLLEMDRAHFFHPSTHLRDHASGELPGRIITGGKGIRIQDSEGREYIDAFAGLYCVNVGYGRTEIADAIYEQAKELAYYHTYVGHSNEPIIELSERIIREWAPAGMSKVYYGMSGSDANETQIKLVWYYNNVLGRPQKKKIISRQRGYHGSGIMTGSLTGLPAFHNHFDLPLEPIRHTTCPHYYRAPAGMSEAEFSRHCADELEKMILAEGPDTVAAFIGEPVMGTGGIVPPPEGYWEAIQAVLNKYDILLIADEVVCGFGRTGSMFGSHHYGMKPDLITVAKGLTSAYAPLSGVIVGEKVWKVLEQGSDQYGPIGHGWTYSGHPICAAAALANLDIIERENLTGNAADTGAYFQQRMRETFGDHPLVGEVRGVGLMAALEFVADKDKRTRFDPSLKVGPRVSAACLEDGMIARAMPHGDILGFAPPLVITRAEVDEIVDIAKQAVDAVADELV

SEQ ID NO: 13 - protein, 426 aa, Escherichia coli
MNSNKELMQRRSQAIPRGVGQIHPIFADRAENCRVWDVEGREYLDFAGGIAVLNTGHLHPKVVAAVEAQLKKLSHTCFQVLAYEPYLELCEIMNQKVPGDFAKKTLLVTTGSEAVENAVKIARAATKRSGTIAFSGAYHGRTHYTLALTGKVNPYSAGMGLMPGHVYRALYPCPLHGISEDDAIASIHRIFKNDAAPEDIAAIVIEPVQGEGGFYASSPAFMQRLRALCDEHGIMLIADEVQSGAGRTGTLFAMEQMGVAPDLTTFAKSIAGGFPLAGVTGRAEVMDAVAPGGLGGTYAGNPIACVAALEVLKVFEQENLLQKANDLGQKLKDGLLAIAEKHPEIGDVRGLGAMIAIELFEDGDHNKPDAKLTAEIVARARDKGLILLSCGPYYNVLRILVPLTIEDAQIRQGLEIISQCFDEAKQ

SEQ ID NO: 14 - DNA, 1365 bp, Pseudomonas sp.
atgacgaaacagaccagcgcccagacccaacactggcaggcgctcagccgcgaacaccacctggctccgttcaccgactacaagcaactgaacgagaagggtgcgcgcatcatcaccaaggccgaaggcgtgtacctgtgggacagcgagggcaacaagatcctcgatggcatggccggcctgtggtgcatgaacgtcggctatggtcgcaaggagcttgccgaggtcgcctacaagcagatgctcgagctgccctactacaacctgttcttccagaccgcgcacccgccagcgctggagctggccaaggccatcgccgacatcgcccccgaaggcatgaaccacgtgttcttcaccggctccggctcggagtccaacgacaccgtgctgcgcatggtccgccactactggagcatcaagggcaagccgcagaagaaggtggtcatcggccgctggaacggctaccacggctccaccgtcgccggcgtcagcctcggcggcatgaaggccctgcacagccagggcgacctgccgatcccgggcatcgtgcacatcgcccagccctactggtacggcgagggcggcgacatgtcggcggaagagttcggcgtctgggccgccgagcagctggagaagaagattctcgaagtcggcgaggagaacgtcgccgccttcatcgccgagccgatccagggcgccggcggcgtgatcgttccgccggacacctactggccgaagatccgcgagatcctcgccaagtacgaaatcctgttcatcgccgacgaagtcatctgcggcttcggccgtaccggcgaatggttcggcagccagtactacggcaacgccccggacctgatgccgatcgccaagggcctgacttccggctacatccccatgggcggcgtaatcgtccgcgacgagatcgtcgataccctgaatgagggtggcgagttctaccacggcttcacttactctggtcacccggtggccgcggcggtggccctggagaacatccgcatcctgcgcgaggagaagatcgtcgagaccgtgaaggcggaaacggcaccttacttgcaaaagcgctggcaggaactggccgaccacccgctggtgggcgaggcccgcggcgtcggcatggtcggcgcgctggagctggtgaagaacaagaagacccgcgagcgcttcgagaacggcgtggggatgctgtgccgcgagcactgcttccgcaacggcctgatcatgcgcgcggtgggagacactatgattatttcgccgccgctggtcatcaccaagccggagatcgacgagttgatcaccctggcgcgcaagtgcctcgatcaaaccgccgccgtagcgctgtcttga

SEQ ID NO: 15 - DNA, 1380 bp, Artificial Sequence
  Molecule produced by ancestral reconstruction
atgaccgattttgatcagctgtttgaacaggatcgtgcccattttatgcatccgagcacccatgcacatgatcacgcaagcggtgcactgcctggtcgtattattaccggtgcaagcggtattcgtattcgcgatcatgaaggtcgtgaactgattgatgcatttgcaggtctgtattgcgtgaatattggttatggtcgtaccgaagttgcagatgcaatctataaacaggcaaaagagctggcctattatcatacctatgttggtcatagcaccgaagccattattgaactgagcagccgtattattcgtgattgggcaccggcaggtatgaaaaaagtttattatggtctgagcggctccgatgcaaatgaaacccagattaaactggtgcgctattataacaatgttctgggtcgtccgcagaaaaaaaaaatcattagccgtcagcgtggttatcatggtagcggtattatgaccggcagcctgaccggtctgccgagctttcatcagcattttgatctgccggttgaaggcattaaacataccgtttgtccgcattggtataaagctccggcaggcatggatgaagcagcatttgttcgttattgtgcagatgaactggaaaaactgattctggcagaaggtccggataccgttgcagcatttattggtgaaccggttatgggtacaggtggtattattgttccgcctaaaggttattgggaagcaattcaggcagtcctgaacaaatatgatgttctgctgattgccgatgaagttgtttgtgcatttggtcgtctgggtagcaaaatgggtagccagcgttatggtatgcgtccggatctgattaccaccgcaaaaggtctgaccagcgcctatgcaccgctgagcgcagttattgttggtgaaaaagtttgggatgtgatcgaaaaagcaagccagaaagaaggtgcaatgggtcatggttggacctatagcggtcatccgatttgtgcagcagcagcactggcaaatctggatattctggaacgtgaaaatctgaccgcaaatgccgcagatgttggtgcatatctgaatcagcgtctgcgtgaaacctttgaaggtcatccgctggttggtgaagttcgtggtgatggtatgctggcagccctggaatttatggcagatcgtgaagcacgtaccccgtttgatccggcactgaaagttggtccgaaagttagcgcagcatgtctggaagatggtatgattgcacgtgccatgccgcatggtgatatcctgggttttgcaccgcctctggttctgacccgtgccgaagcagatgaaattgttggtattgcaaaagcagccgttgatgaagtggcaggcgaagttctgtaa

SEQ ID NO: 16 - DNA, 1380 bp, Artificial Sequence
  Molecule produced by ancestral reconstruction
atgaccgatctggatcagctgtttgaaatggatcgtgcacattttatgcatccgagcacccatgcacatgatcacgcaagcggtgcactgcctggtcgtattattaccggtggtaaaggtattcgtatccaggatcatgaaggtcgcgaatatattgatgcatttgcaggtctgtattgcgtgaatattggttatggtcgtaccgaagttgcagatgcaatttatgaacaggcaaaagagctggcctattatcatacctatgttggtcatagcaccgaagccattattgaactgagcagccgtattattcgtgattgggcaccggcaggtatgaaaaaagtttattatggtatgagcggtagcgacgcaaatgaaacccagattaaactggtgtggtattacaataatgttctgggtcgtccgcagaaaaaaaaaatcattagccgtcagcgtggttatcatggtagcggtattatgaccggcagcctgaccggtctgccgagctttcatcagcattttgatctgccgattgaacgcattaaacataccgtttgtccgcactggtataaagctccggcaggcatgagcgaagcacagtttgttcgttattgtgcagatgaactggaaaaactgattctggcagaaggtccggataccgttgcagcatttattggtgaaccggttatgggtacaggtggtattattccgcctccgcagggttattgggaagcaattcaggcagttctgaacaaatatgatattctgctgattgccgatgaagtggtttgtggttttggtcgtctgggtagcaaaatgggtagccagcattatggcatgaaaccggatctgattaccgttgccaaaggtctgaccagcgcctatgcaccgctgagtggtgttattgttggtgaaaaagtttgggacgtgattgaaaaaggtagccaagaacatggtccgatgggtcatggttggacctatagcggtcatccgatttgtgcagcagcagcactggcaaatctggatattctggaacgtgaaaatctgaccggtaatgcagcagatgttggtgcatatctgcagcagcgtctgcgtgaaacctttggtggtcatccgctggttggtgaagttcgtggtgttggtatgctggcagccctggaatttatggcagataaagatgcacgtaccccgtttgatcctgccctgaaagttggtccgaaagttagcgcagcatgtctggaagatggtatgattgcacgtgccatgccgcatggtgatatcctgggttttgcaccgcctctggttattacccgtgccgaagttgatgaaattgttggtattgcaaaacaggccgttgatgaagttgcggatgaagttctgtaa

SEQ ID NO: 17 - DNA, 1380 bp, Artificial Sequence
  Molecule produced by ancestral reconstruction
atgcagagcctggatcagctgtttgaaatggatcgtgcacattttatgcatccgagcacccatgcacatgatcacgcaagcggtgcactgcctggtcgtattattaccggtggtaaaggtattcgcatcgaagatcatgaaggtcgcgaatatattgatgcatttgcaggtctgtattgcgtgaatattggttatggtcgtaccgaagttgcagatgcaatttatgaacaggcaaaacagctggcctattatcatacctatgttggtcatagcaccgatgccattattgaactgagcagccgtattattcgtgattgggcaccggcaggtatgaaaaaagtttattatggtatgagcggtagcgacgcaaatgaaacccagattaaactggtgtggtattacaataatgttctgggtcgtccgaacaaaaaaaaaatcattagccgtgaacgcggttatcatggtagcggtattgtgaccggtagcctgaccggtctgccgagctttcatcagcattttgatctgccgattgatcgtgttaaacataccgtttgtccgcattggtatcgtgctccggcaggcatgagcgaagcacagtttgttgcatattgtgttgaggaactggaaaaactgattgcacgtgaaggtgcagataccattgcagcatttattgcagaaccggttatgggtacaggtggtattattccgcctccgcagggttattgggaagcaattcaggcagttctgcgtaaacatgatattctgctgattgccgatgaagttgtttgtggttttggtcgtctgggtagcaaaatgggtagccagcattatggcattaaaccggatctgattaccgttgcaaaaggtctgaccagcgcctatgcaccgctgagtggtgttattgttggtgaaaaagtttgggacgtgattgaaaaaggtagccaagaacatggtccgatgggtcatggttggacctatagcggtcatccgatttgtgcagcagcagcactggcaaatctggatattctggaacgtgaaaatctgaccggtaatgcagcagatgttggtgcatatctgcagcagcgtctgcatgaagcatttggtgcacatccgctggttggtgaagttcgtggtgttggtatgctggcagccctggaatttatggcagataaagatgcacgtaccccgtttgatccggcactgaaagttggtccgaaagttagcgcagccgcactggaagatggtatgattgcccgtgcaatgccgcatggtgatatcctgggttttgcaccgcctctggttaccacccgtgccgaagttgatgaaattgttggtattgcaaaacaggccgttgatgaagtggcagatgaagttctgtaa

SEQ ID NO: 18 - DNA, 1380 bp, Artificial Sequence
  Molecule produced by ancestral reconstruction
atgaccagcctggatcagctgtttgaagaagatcgcgcacattttatgcatccgagcacccatgcacatgatcacgcaagcggtgcactgcctggcaaaattgttaccggtggtaaaggtattcgcatcgaagatcatcagggtcgcgaatatattgatgcatttgcaggtctgtattgcgtgaatattggttatggtcgtaccgaagttgcagatgcaatttatgaacaggcaaaacagctggcctattatcatacctatgttggtcatagcaccgatgccattattgaactgagcagccgtattattcgtgattgggcaccggcaggtatgaaaaaagtttattatggtatgagcggtagcgacgcaaatgaaacccagattaaaatcgtgtggtactacaataatgttctgggtcgtccgaacaaaaaaaaaatcattagccgtgaacgcggttatcatggtagcggtattgtgaccggtagcctgaccggtctgccgagctttcatcagcattttgatctgccgattgatcgtgttaaacataccgtttgtccgcactggtataaagctccggcaggcatgagcgaagcacagtttgttgcatattgtgttgaggaactggaaaaactgattgcacgtgaaggtgcagataccattgcagcatttattgcagaaccggttatgggtacaggtggtattattgcaccgcctcagggttattgggaagcaattcaggcagttctgcgtaaacatgatattctgctgattagtgatgaagtggtttgtggttttggtcgtctgggtagcaaaatgggtgcacagcattatggcattaaaccggatctgattaccgttgcaaaaggtctgaccagcgcctatgcaccgctgagtggtgttattgttggtgaaaaagtttgggacgtgattgaaaaaggtagccaagaacatggtccgatgggtcatggttggacctatagcggtcatccgatttgtgcagcagcagcactggcaaatctggatattctggaacgtgaaaatctgaccggtaatgcagcagatgttggtgcatatctgcagcagcgtctgcatgaagcatttggtgcacatccgctggttggtgaagttcgtggtgttggtatgctggcagccctggaatttatggcagataaagatgcacgtaccccgtttgatcctgccctgaaagttggtccgaaagttagcgcagccgcactggaagatggtgtgattgcccgtgcaatgccgcatggtgatatcctgggttttgcaccaccgctggttaccacccgtgccgaagttgatgaaattgttggtattgttaaacaggccgttgatgaagttgccgatgaagttctgtaa

SEQ ID NO: 19 - DNA, 1380 bp, Artificial Sequence
  Molecule produced by ancestral reconstruction
atgaccagcctggaacagctgctggaaatggatcgtgcacattttatgcatccgagcacccatgcctatgatcatgcaagcggtgcactgcctggtcgtattattaccggtggtaaaggtattcgcatcgaagatcatgaaggtcgcgaatatattgatgcatttgcaggtctgtattgcgtgaatattggttatggtcgtgaagaggttgcagatgcaatttatgaacaggcaaaacagctggcctattatcatacctatgttggccatagcaatgatccggttattgaactgagcagccgtattattgaagattgggcaccggcaggtatgaaaaaagttttttatggtatgagcggtagcgacgcaaatgaaacccagattaaactggtgtggtattacaataatgttctgggtcgtccgaacaaaaaaaaaatcattgcacgtgaacgcagctatcatggtagcggtattgtgaccggtagcctgaccggtctgccgagctttcatcagcattttgatctgccgattgatcgtgttaaacataccgtttgtccgcattggtataatgcacctccgggtatgagtgaagcacagtttgttgcatattgtgtggaagaactggaaaaactgattgcccgtgaaggtgcagataccattgcagcatttattgcagaaccggttatgggtacaggtggtattgttccgcctccgcagggttattgggaagcaattcaggcagttctgcgtaaacatgatattctgctgattgccgatgaagttgtttgtggttttggtcgtctgggtagcaaaaccggtagcgaacattatggtattaaaccggatctgattaccgttgcaaaaggtctgaccagcgcctatgcaccgctgagcgcagttattgttagcgaaaaagtttgggatgtgatcgaaaaaggtagccgtgaacatggtgttatgggtcatggttggacctatagcggtcatccggtttgtgcagcagcagcactggcaaatctggatattctggaacgtgaaaatctgaccggtaatgcagcagatgttggtgcatatctgcagcagcgtctgcatgaagcatttggtgcacatccgctggttggtgaagttcgtggtgttggtatgctggcagccctggaatttatggcagataaaggtgcacgtaccccgtttgatccggcactgaaagttagccagaaagttgcagccgcagcgctggaagatggtctgattgttcgtgccctgccgcatggtgatatcctgggttttgcaccgcctctggttaccacccgtgccgaagttgatgaaattgttgcaattgccaaagaagcctttgacgaagttgcggatgcagttctgtaa

SEQ ID NO: 20 - DNA, 1380 bp, Artificial Sequence
  Molecule produced by ancestral reconstruction
atgaccgataccgatgatctgctggaaatggatcgtgcacatttttttcatccgagcacccatctgcgtgatcatgcaagcggtgaactgcctggtcgtattattaccggtggtaaaggtattcgtattcaggatagcgaaggtcgcgaatatattgatgcatttgcaggtctgtattgcgtgaatgttggttatggtcgtaccgaaattgcagatgccatttatgaacaggcaaaagagctggcctattatcatacctatgttggccatagcaacgaaccgattattgaactgagcgaacgtattattcgtgaatgggcaccggcaggtatgagcaaagtttattatggtatgagcggttccgatgcaaatgaaacccagattaaactggtgtggtattacaataatgttctgggtcgtccgcagaaaaaaaaaatcattagccgtcagcgtggttatcatggtagcggtattatgaccggcagcctgaccggtctgcctgcatttcataatcattttgatctgccgctggaaccgattcgtcataccacctgtccgcattattatcgtgctccggcaggcatgtcagaagcagaatttagtcgtcattgtgcagatgagctggaaaaaatgattctggcagaaggtccggataccgttgcagcatttattggtgaaccggttatgggtacgggtggtattgttccgcctccggaaggttattgggaagcaattcaggccgttctgaacaaatatgatattctgctgattgccgatgaagtggtttgtggttttggtcgcaccggtagcatgtttggtagccatcattatggcatgaaaccggatctgattaccgttgccaaaggtctgaccagcgcctatgcaccgctgagtggtgttattgttggtgaaaaagtttggaaagttctggaacagggtagcgatcagtatggtccgattggtcatggttggacctatagcggtcatccgatttgtgcagcagcagcactggcaaatctggatattatcgaacgtgaaaatctgaccggtaatgcagcagataccggtgcctattttcagcagcgtatgcgtgaaacctttggtgatcatccgctggttggtgaagttcgtggtgttggtctgatggcagccctggaatttgttgcagataaagataaacgtacccgttttgatccgagcctgaaagttggtccgcgtgttagcgcagcatgtctggaagatggtatgattgcacgtgccatgccgcatggtgatatcctgggttttgcaccgcctctggttattacccgtgccgaagttgatgaaattgtggatattgcaaaacaggcagttgatgccgttgcggatgaactggtgtaa